(19) United States
(12) Patent Application Publication
(10) Pub. No.: US 2025/0387554 A1
(43) Pub. Date: Dec. 25, 2025

(54) Medical Waste Collection Systems, Manifolds, And Related Methods

(71) Applicant: Stryker Corporation, Portage, MI (US)

(72) Inventors: Sachin Jain, Gurgaon (IN); Grant Westphal, Delton, MI (US); Brian MacLachlan, Norton Shores, MI (US); Brandon Lee Laure, Satellite Beach, FL (US)

(73) Assignee: Stryker Corporation, Portage, MI (US)

(21) Appl. No.: 19/248,366

(22) Filed: Jun. 24, 2025

Related U.S. Application Data
(60) Provisional application No. 63/663,243, filed on Jun. 24, 2024.

(51) Int. Cl. A61M 1/00; G06K 19/04

(52) U.S. Cl. CPC A61M 1/74; A61M 1/60; G06K 19/045

(57) ABSTRACT

A medical waste collection system includes a medical waste collection device for providing suction at a surgical site, and a manifold releasably couplable to the medical waste collection device. The manifold defines a pathway through which the medical waste collection device is configured to provide suction to the surgical site. The medical waste collection device is configured to control activation of suction based on a proper manifold being present.

### RELATED APPLICATION(S)

This application claims priority to and all the benefits of U.S. Provisional Patent Appl. No. 63/663,243, filed Jun. 24, 2024, the entire contents of which are hereby incorporated by reference herein.

### BACKGROUND

A byproduct of some surgical procedures is the generation of liquid, semisolid, and/or solid waste material. The liquid waste material may include bodily fluids and irrigating solution(s) at the surgical site, and the solid and semisolid waste material may include bits of tissue and pieces of surgical material(s). The medical waste, regardless of its phase, is preferably collected so it neither fouls the surgical site nor becomes a biohazard in the medical suite in which the procedure is being performed.

The medical waste may be removed from the surgical site through a suction tube under the influence of a vacuum provided by a medical waste collection system. One exemplary medical waste collection system is sold under the tradename NEPTUNE by Stryker Corporation (Kalamazoo, Mich.), with certain versions of the medical waste collection system disclosed in commonly owned United States Patent Publication No. 2005/0171495, published Aug. 4, 2005, International Publication No. WO 2007/070570, published Jun. 21, 2007, and International Publication No. WO 2014/066337, published May 1, 2014, the entire contents of each of which are incorporated herein by reference.

Manifolds may be provided that facilitate interfacing the suction tube with the medical waste collection system. Authentic manifolds may include an intricate set of features and undergo stringent manufacturing and quality controls configured to provide optimized operation of a given medical waste collection system, and to avoid clogging or compromise of components of the given medical waste collection system. It is thus in the interest of both patients and medical personnel alike that manifolds used with a given medical waste collection system are authentic.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a medical waste collection system including a medical waste collection device for providing suction at a surgical site. The system also includes a manifold couplable to the medical waste collection device, the manifold defining a pathway through which the medical waste collection device is configured to provide the suction to the surgical site, and the manifold including at least one RFID tag with at least one memory device storing first and second manifold authentication data. The medical waste collection device includes a waste container for collecting medical waste material; a receiver in fluid communication with the waste container and dimensioned to removably receive the manifold; at least one reader disposed adjacent the receiver and configured to communicate with the at least one RFID tag according to a first communications protocol and a second communications protocol when the manifold is inserted in the receiver; and at least one controller coupled to the at least one reader. The at least one controller is configured to operate the at least one reader according to the first communications protocol to read the first manifold authentication data from the at least one RFID tag; operate the at least one reader according to the second communications protocol to read the second manifold authentication data from the at least one RFID tag, and control operation of the medical waste collection device to provide suction based on the read first and second manifold authentication data. Other general aspects include a corresponding method and a corresponding computer system, apparatus, and computer program recorded on one or more computer storage devices, each configured to perform the actions of the method.

Another general aspect includes a medical waste collection system including a medical waste collection device for providing suction at a surgical site. The system also includes a manifold couplable to the medical waste collection device, the manifold defining a pathway through which the medical waste collection device is configured to provide the suction to the surgical site, and including at least one RFID tag having a first memory device and a second memory device, the first and second memory devices storing first and second manifold authentication data respectively. The medical waste collection device includes a waste container for collecting medical waste material; a receiver in fluid communication with the waste container and dimensioned to removably receive the manifold; at least one reader disposed adjacent the receiver and configured to communicate with the at least one RFID tag when the manifold is inserted in the receiver; and at least one controller coupled to the at least one reader and configured to: operate the at least one reader to read the first manifold authentication data from the first memory device of the at least one RFID tag; operate the at least one reader to read the second manifold authentication data from the second memory device of the at least one RFID tag; combine the first and second manifold authentication data to form an originality signature; and control operation of the medical waste collection device to provide suction based on the combined originality signature. Other general aspects include a corresponding method and a corresponding computer system, apparatus, and computer program recorded on one or more computer storage devices, each configured to perform the actions of the method.

Another general aspect includes a medical waste collection system including a medical waste collection device for providing suction at a surgical site. The system also includes a manifold couplable to the medical waste collection device, the manifold defining a pathway through which the medical waste collection device is configured to provide the suction to the surgical site, and including an RFID tag. The medical waste collection device includes a waste container for collecting medical waste material; a receiver in fluid communication with the waste container and dimensioned to removably receive the manifold; at least one reader disposed adjacent the receiver and configured to communicate with the RFID tag when the manifold is inserted in the receiver; and at least one controller configured to operate the at least one reader to obtain authentication data from the RFID tag and to control operation of the medical waste collection device to provide suction based on the obtained authentication data. The RFID tag is configured to identify operation of the at least one reader according to a first communications protocol; transmit first authentication data to the at least one reader based on the identification of the first communications protocol; identify operation of the at least one reader according to a second communications protocol; and transmit second authentication data, different from the first authentication data, to the at least one reader based on the identification of the second communications protocol. Other general aspects include a corresponding method and a corresponding computer system, apparatus, and computer program recorded on one or more computer storage devices, each configured to perform the actions of the method.

Another general aspect includes a medical waste collection system including a medical waste collection device for providing suction at a surgical site. The system also includes a manifold couplable to the medical waste collection device, the manifold defining a pathway through which the medical waste collection device is configured to provide the suction to the surgical site, and including an RFID tag. The medical waste collection device includes a waste container for collecting medical waste material; a receiver in fluid communication with the waste container and dimensioned to removably receive the manifold; at least one reader disposed adjacent the receiver and configured to communicate with the RFID tag when the manifold is inserted in the receiver; and at least one controller configured to operate the at least one reader to obtain authentication data from the RFID tag and to control operation of the medical waste collection device to provide suction based on the obtained authentication data. The RFID tag is configured to: responsive to receiving a first request from the at least one reader, communicate first authentication data to the at least one reader, the first authentication data being generated based on a first authentication datum; identify occurrence of a predefined event; and responsive to receiving a second request from the at least one reader after the predefined event, communicate second authentication data to the at least one reader, the second authentication data being generated based on a second authentication datum different from the first authentication datum. Other general aspects include a corresponding method and a corresponding computer system, apparatus, and computer program recorded on one or more computer storage devices, each configured to perform the actions of the method.

Other general aspects include the manifold of any of the above aspects, a method for preparing the manifold of any of the above aspects, the RFID tag(s) of any of the above aspects, a method for preparing the RFID tag(s) of any of the above aspects, and a medical waste collection device of any of the above aspects.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a medical waste collection system including a medical waste collection device and two manifolds each configured to be removably inserted into a different receiver of the medical waste collection device.

FIG. 2 is a perspective view of the manifold and receiver with the manifold oriented for insertion into an opening of the receiver, with the manifold including an RFID tag for authentication of the manifold and the receiver including an RFID reader for communicating with the RFID tag.

FIGS. 3A and 3B are sectional views of the manifold and receiver with the manifold including multiple RFID tags for authentication of the manifold and the receiver including multiple RFID readers for communicating with the RFID tags.

FIGS. 4A and 4B are sectional views of the manifold and receiver with the manifold including a single RFID tag for authentication of the manifold and the receiver including media for shaping interrogation signals of the RFID reader of the receiver.

FIGS. 5A and 5B are sectional views of the manifold and receiver with the manifold including multiple RFID tags for authentication of the manifold and the receiver including media for shaping interrogation signals of the RFID reader of the receiver.

FIG. 6 is a schematic view of a manifold with multiple RFID tags.

FIG. 7 is a schematic view of a manifold with a single RFID tag.

FIG. 8 is a flowchart of a method for authenticating a received manifold that may be performed by the medical waste collection device.

FIG. 9 is a flowchart of a method for operating one or more RFID tags of a manifold received by the medical waste collection device to facilitate authentication of the manifold by the medical waste collection device.

### DETAILED DESCRIPTION

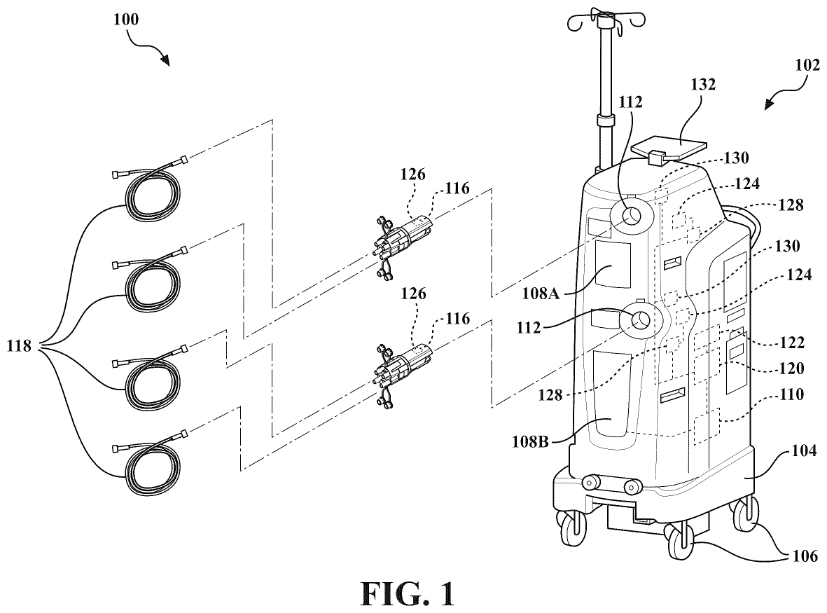

FIG. 1 illustrates an exemplary configuration of a medical waste collection system 100, such as a surgical waste collection system, for collecting waste material generated during medical procedures. The medical waste collection system 100 may collect and/or store the waste material until it is necessary or desired to off-load and dispose of the waste material. To this end, the medical waste collection system 100 may include a medical waste collection device 102 (also referred to herein as a “rover”) for collecting and storing the waste material at the location of the medical procedure. The rover 102 (i.e., medical waste collection device) may be mobile so as to thereafter enable easy transportation of the rover 102 to a docking station through which the waste material is then emptied.

The rover 102 may include a base 104 and wheels 106 for moving the rover 102 along a floor surface within a medical facility. The rover 102 may also include at least one waste container 108 defining a waste volume for collecting and storing the waste material. FIG. 1 shows the rover 102 with a first waste container 108A arranged above a second waste container 108B having a relatively greater or larger volume than the first waste container 108A. A vacuum pump 110 (shown in phantom) may be supported on the base 104 and configured to draw suction on one or both of the first and second waste containers 108A, 108B through one or more vacuum lines internal to the rover 102. Suitable construction and operation of several subsystems of the rover 102 may be disclosed in the aforementioned, commonly owned United States Patent Publication No. 2005/0171495, International Publication No. WO 2007/070570, and International Publication No. WO 2014/066337. Suitable construction and operation of several subsystems of the rover 102 may also be disclosed in commonly owned International Publication No. WO 2017/112684, published Jun. 29, 2017, the entire contents of which are hereby incorporated herein by reference.

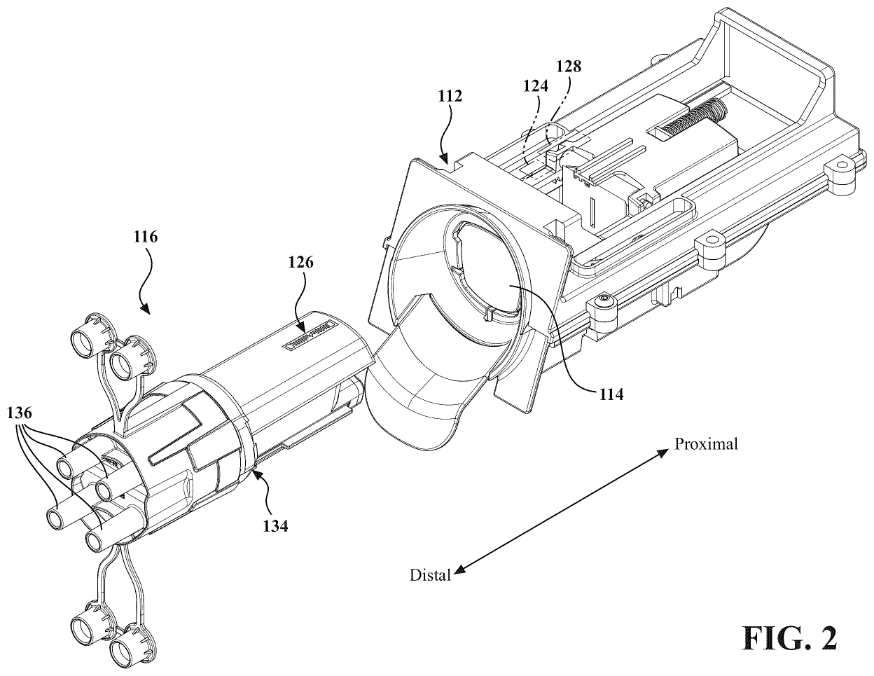

The rover 102 may further include at least one receiver 112 supported on the base 104. In a general sense, each receiver 112 may define an opening 114 (see FIG. 2) dimensioned to removably receive at least a portion of a manifold 116, such as a surgical waste collection manifold, in a manner to be described throughout the present disclosure. FIG. 1 shows two receivers 112 each associated with a respective one of the waste containers 108A, 108B. Alternatively, a single receiver 112 may be provided for both waste containers 108A, 108B. Each receiver 112 may include a suction inlet configured to be arranged in fluid communication with at least one of the waste containers 108A, 108B. A suction path may be established from at least one suction tube 118 to the waste containers 108A, 108B through the manifold(s) 116 removably inserted into the receiver(s) 112. The vacuum generated by the vacuum pump 110 may be drawn on the suction tube(s) 118, and the waste material at the surgical site may be drawn through the manifold(s) 116, through the suction inlet of the receiver(s) 112, and into the waste containers 108A, 108B.

The rover 102 may additionally include a rover controller 120. The rover controller 120 may be configured to control actuation of the rover 102. To this end, the rover controller 120 may be in communication with the vacuum pump 110 and may provide for overall control of the rover 102. For instance, the rover controller 120 may regulate the on/off operation of the vacuum pump 110 and may also regulate the vacuum flow through the manifold(s) 116. The rover controller 120 may be in communication with a memory device 122 of the rover 102. The memory device 122 may store data and computer-executable instructions for authenticating an inserted manifold 116, as described in more detail below.

The rover 102 may further include a user interface 132 in operable communication with the rover controller 120. The user interface 132 may be configured to present operational data, accept user inputs, and provide audible tones to a user. For instance, the user interface 132 may include a touchscreen display and a speaker. Surgical personnel may enter commands to regulate the rover 102 through the user interface 132, such as by pressing button images presented on the user interface 132.

Still referring to FIG. 1, the rover 102 may also include at least one reader 124 positioned adjacent each receiver 112. The reader(s) 124 positioned adjacent each receiver 112 may be configured to communicate with one or more RFID tags 126 of the manifold 116 when the manifold 116 is inserted into the receiver 112. The RFID tag(s) 126 may be coupled to one or more surfaces, such as one or more internal and/or external surfaces, of the manifold 116. The rover controller 120 may be in communication with each reader 124, such as through a reader controller 128 coupled to the reader 124.

In one implementation, the rover controller 120 may be configured to instruct the reader(s) 124 associated with each receiver 112 to repetitively emit basic interrogation signals for the RFID tag(s) 126. If a manifold 116 is not seated in a given receiver 112, the reader(s) 124 associated with the given receiver 112 may not receive a responsive communication to the interrogation signals, in which case the rover controller 120 may determine that a manifold 116 is not currently seated in the given receiver 112.

The rover controller 120 may be configured to inhibit activation of the vacuum pump 110 until a manifold 116 has been seated into the receiver 112 and authenticated by the rover controller 120. An authentic manifold 116 may be designed with a specific set of features and subject to stringent manufacturing and quality standards so as to provide optimized operation of the medical waste collection system 100. Such a manifold 116 may also have a limited lifespan. Use of a manifold with the rover 102 that lacks such features or is of lesser quality, or use of a manifold beyond its limited lifespan, may adversely affect the performance of the medical waste collection system 100. For instance, such use may result in clogging or other compromise of the components of the medical waste collection system 100, potentially causing reduced suction and/or contamination. The rover controller 120 may thus serve as a master override that prohibits the vacuum pump 110 from being actuated unless, as described below, an appropriate manifold 116 is fitted to the rover 102. If the user tries to actuate the vacuum pump 110 without such a manifold 116 being inserted, the rover controller 120 may be configured to prevent activation of the vacuum pump 110. The rover controller 120 may also be configured to cause a warning message to be presented on the user interface 132.

Each manifold 116 and receiver 112 may be configured such that, when the manifold 116 is seated in the receiver 112, the reader(s) 124 associated with the receiver 112 are within communication range of the RFID tag(s) 126 of the manifold 116. Once this event occurs, the RFID tag(s) 126 may send a basic response to the interrogation signals sent by the reader(s) 124, which may be forwarded to the rover controller 120 as an indication that a manifold 116 has been inserted into the receiver 112. The rover controller 120 may then be configured to obtain authentication data from the RFID tag(s) 126 through the reader(s) 124 (and potentially through the reader controller(s) 128 coupled to the reader(s) 124). The rover controller 120 may thereafter be configured to perform an authentication procedure based on the received data as described in more detail below.

FIG. 2 illustrates a manifold 116 and receiver 112 prior to insertion of the manifold 116 into the receiver 112 and/or after removal of the manifold 116 from the receiver 112. The manifold 116 may be configured to be inserted into the receiver 112 through the opening 114, and the suction tube(s) 118 may be configured to be removably coupled to inlet fitting(s) 136 of the manifold 116. The resulting arrangement is schematically reflected in FIG. 1, in which two suction tubes 118 are coupled to two of four inlet fittings 136 of each of the manifolds 116. Any number of inlet fittings 136 are contemplated, and it is further contemplated that the suction tube(s) 118 may be integral with a housing 134 of each manifold 116. The aforementioned suction path may thus be established, and an instrument (not shown) coupled to an end of the suction tube(s) 118 opposite the manifold(s) 116 may be directed to the surgical site to collect the waste material under the influence of the vacuum provided by the vacuum pump 110.

As described above, the reader(s) 124 of the rover 102 may cooperate with the RFID tag(s) 126 of a given manifold 116 to exchange authentication data and track usage of the manifold 116. To this end, the rover controller 120 may be configured to operate the reader(s) 124 to communicate various commands such as read requests to the RFID tag(s) 126, which in turn may be configured to determine whether the communications were sent according to the expected communications protocols prior to processing such requests. In other words, responsive to receiving a communication including a command that is according to a communications protocol not associated with the command, the RFID tag(s) 126 may be configured to reject the request. As an example, in some implementations, a given command may be associated with varying communications protocols such that, responsive to receiving a first communication associated with the command from the reader(s) 124 according to a first of the associated communications protocols, prior to processing the command, the RFID tag(s) 126 may be configured to determine whether a further communication corresponding to the command is received according to a second of the associated communications protocols, such as within a predefined time period of the first communication. If not, then the RFID tag(s) 126 may be configured to reject the command, and may also be configured to render itself inoperable, such as to no longer be authenticatable for use with a rover 102.

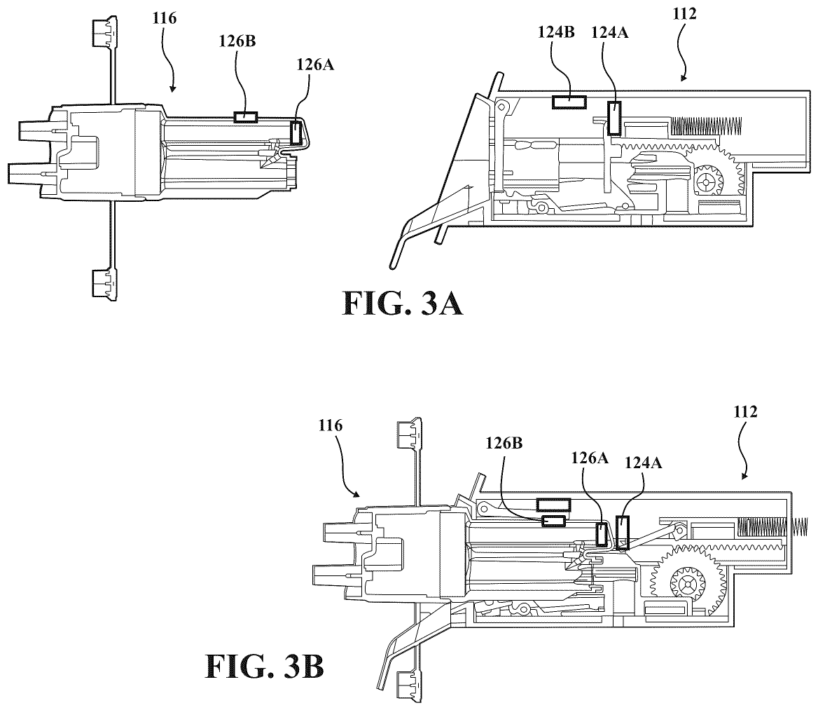

Various exemplary implementations of a manifold 116 and receiver 112 are illustrated in FIGS. 3A-5B. FIGS. 3A and 3B depict an implementation in which the manifold 116 includes two RFID tags 126, such as a primary RFID tag 126A and a secondary RIFD tag 126B, and the receiver 112 correspondingly includes two readers 124, such as a primary RFID reader 124A and a secondary RFID reader 124B. As illustrated in FIG. 3B, the RFID tags 126A, 126B and readers 124A, 124B may be arranged such that the primary RFID tag 126A aligns with the primary reader 124A and the secondary RFID tag 126B aligns with the secondary reader 124B when the manifold 116 is seated in the receiver 112. Each reader 124A, 124B may be configured to communicate with the RFID tag 126A, 126B aligned with the reader 124A, 124B upon insertion of the manifold 116. In some implementations, each RFID tag 126A, 126B and reader 124A, 124B pair may be configured to communicate via a different communications protocol so as to reduce communication interference between one another.

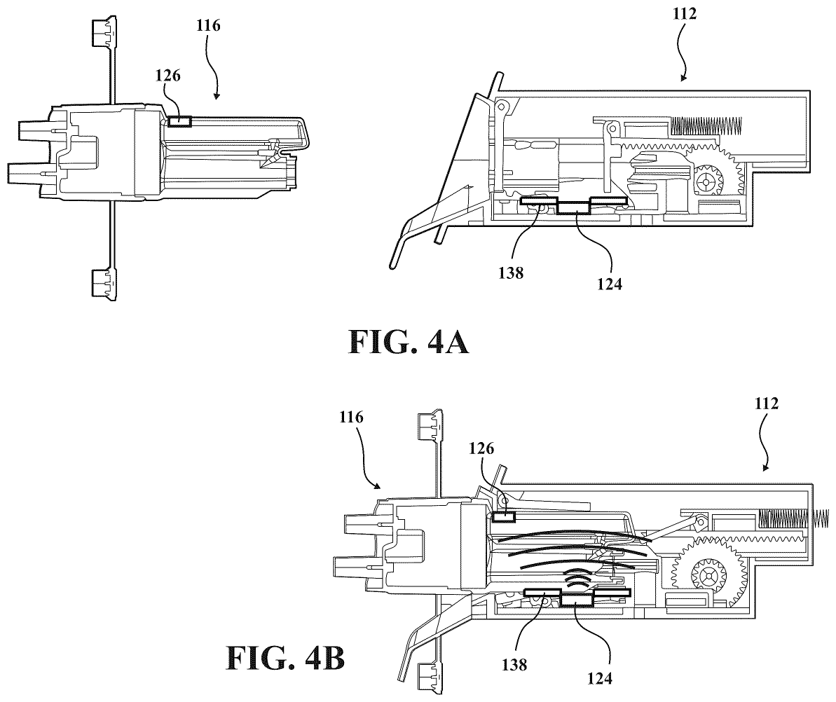

FIGS. 4A and 4B depict another implementation in which the manifold 116 includes a single RFID tag 126 and the receiver 112 includes a single reader 124. As illustrated in FIG. 4B, the RFID tag 126 and reader 124 may be arranged such that the RFID tag 126 is offset from the reader 124 when the manifold 116 is seated in the receiver 112. In some implementations, the rover controller 120 may be configured to authenticate the manifold 116 based on the offset between the reader 124 and the RFID tag 126.

Specifically, the rover controller 120 may be configured to operate the reader 124 at varying power levels. Due to the offset, the RFID tag 126 may be configured to not respond to interrogation signals from the reader 124 when the power level is less than a threshold power level. Thus, responsive to the reader 124 receiving a response when the power level is less than the threshold power level, the rover controller 120 may be configured to determine that an authentic manifold 116 is not inserted in the receiver 112. Conversely, responsive to the reader 124 not receiving a response when the power level is less than the threshold power level and receiving a response when the power level is greater than or equal to the threshold power level, or alternatively greater than or equal to another higher threshold power level, the rover controller 120 may be configured to determine that an authentic manifold 116 is inserted in the receiver 112.

In some implementations, at least one of the manifold 116 and the receiver 112 may include a layer of blocking media 138 adjacent the RFID tag 126 and the reader 124, respectively. When the manifold 116 is seated in the receiver 112, the layer of blocking media 138 may be configured to interfere, attenuate, and/or shape communications from the RFID tag 126 and/or the reader 124. To this end, the layer of blocking media 138 may be configured to redirect signals generated by the reader 124 toward the RFID tag 126, and/or vice versa, such as to improve communication between the RFID tag 126 and the reader 124. For example and without limitation, the layer of blocking media 138 may be formed from copper, aluminum, nickel, or an alloy thereof.

In some implementations, the layer of blocking media 138 may be configured to interfere, attenuate, and/or shape communications according to one or more communications protocols more than communications according to one or more other communications protocols. In this way, the layer of blocking media 138 may assist the rover controller 120 with determining the authenticity of an inserted manifold 116, such as by interfering with certain communications between the reader 124 and RFID tag 126 and permitting others. More specifically, the layer of blocking media 138 may be arranged to permit communications between the reader 124 and the RFID tag 126 sent according to one or more communications protocol(s) (e.g., protocols with relative high power levels and/or relatively low carrier frequencies), and block or attenuate communications between the reader 124 and the RFID tag 126 sent according to one or more other communications protocol(s) (e.g., protocols with relatively low power levels and/or relatively high carrier frequencies).

The rover controller 120 may be configured to operate the reader 124 according to both the blocked or attenuated communications protocols and the permitted communications protocols. Responsive to the reader 124 receiving a response from the RFID tag 126 when operating according to one of the blocked or attenuated communications protocols, and/or not receiving a response from the RFID tag 126 when operating according to one of the permitted communications protocols, the rover controller 120 may be configured to determine that an authentic manifold 116 is not inserted in the receiver 112. Conversely, responsive to the reader 124 not receiving a response from the RFID tag 126 when operating according to one of the blocked or attenuated communications protocols, and/or receiving a response from the RFID tag 126 when operating according to one of the permitted communications protocols, the rover controller 120 may be configured to determine that an authentic manifold 116 is inserted in the receiver 112.

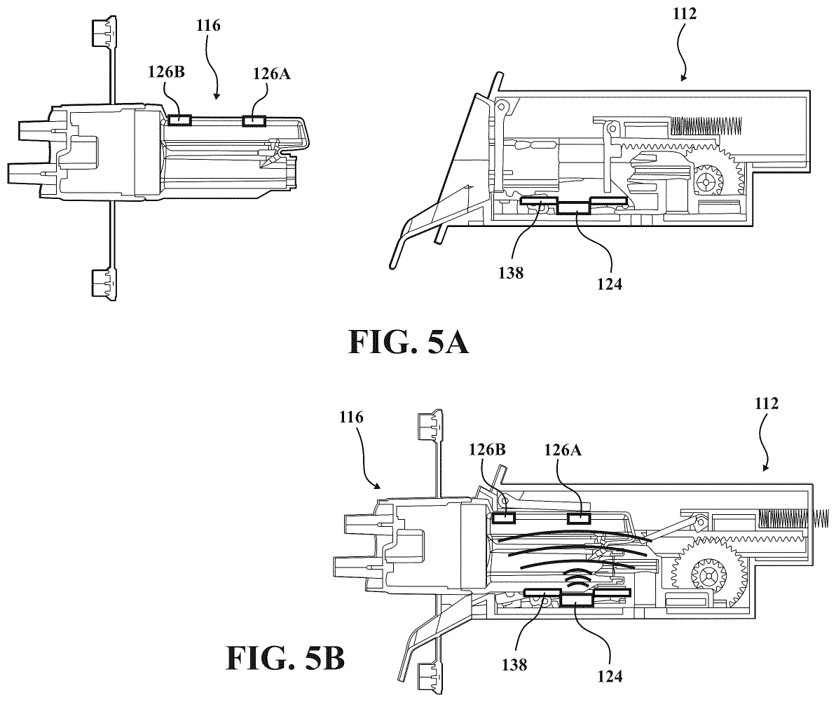

FIGS. 5A and 5B depict a further implementation in which the manifold 116 includes two RFID tags 126, such as a primary RFID tag 126A and a secondary RFID tag 126B, and the receiver 112 includes a single reader 124. As illustrated in FIG. 5B, the RFID tags 126A, 126B and reader 124 may be arranged such that the secondary RFID tag 126B is offset from the reader 124 when the manifold 116 is seated in the receiver 112, and the primary RFID tag 126A is nearer to and/or aligned with the reader 124 when the manifold 116 is seated in the receiver 112. Similar to the previous example, in some implementations, the rover controller 120 may be configured to authenticate the manifold 116 based on the distinct positions of the RFID tags 126A, 126B relative to the reader 124.

Specifically, the rover controller 120 may be configured to operate the reader 124 at varying power levels. Due to the positions of the RFID tags 126A, 126B relative to the reader 124, the primary RFID tag 126A and not the secondary RFID tag 126B may be configured to respond to interrogation signals from the reader 124 when the power level is less than a threshold power level. Thus, the rover controller 120 may be configured to determine that an authentic manifold 116 is not received in the receiver 112 responsive to occurrence of at least one of the following: the reader 124 receiving a response from the secondary RFID tag 126B when the power level is less than the threshold power level; the reader 124 not receiving a response from the primary RFID tag 126A when the power level is less than the threshold power level and greater than a further, lower threshold power level; the reader 124 not receiving a response from either of the RFID tags 126A, 126B when the power level is greater than the threshold power level; and the reader 124 receiving a response from either the RFID tags 126A, 126B when the power level is less than the further, lower threshold power level. Conversely, the rover controller 120 may be configured to determine that an authentic manifold 116 is received in the receiver 112 responsive to occurrence of at least one of the following: the reader 124 receiving a response from both the RFID tags 126A, 126B when the power level is greater than the threshold power level; the reader 124 receiving a response from the primary RFID tag 126A and not from the secondary RFID tag 126B when the power level is less than the threshold power level and greater than the further, lower threshold level; and the reader 124 not receiving a response from either of the RFID tags 126A, 126B when the power level is less than the further, lower threshold power level.

In some implementations, similar to the previous example, at least one of the manifold 116 and the receiver 112 may include a layer of blocking media 138 between the RFID tags 126A, 126B and adjacent the reader 124, respectively. When the manifold 116 is seated in the receiver 112, the layer of blocking media 138 may be configured to interfere, attenuate, and/or shape communications from the RFID tags 126A, 126B and/or the reader 124. To this end, the layer of blocking media 138 may be configured to redirect signals generated by the reader 124 toward the RFID tags 126A, 126B, and/or vice versa, such as to improve communication between the RFID tags 126A, 126B and the reader 124.

Further similar to the previous example, in some implementations, the layer of blocking media 138 may be configured to interfere, attenuate, and/or shape communications according to one or more communications protocols more than communications according to one or more other communications protocols. In this way, the layer of blocking media 138 may assist the rover controller 120 in determining the authenticity of an inserted manifold 116, such as by interfering with certain communications between the reader 124 and the RFID tags 126A, 126B and permitting others. More specifically, the layer of blocking media 138 may be arranged to permit communications between the reader 124 and at least one of the RFID tags 126A, 126B sent according to one or more communications protocol(s) (e.g., protocols with relative high power levels and/or relatively low carrier frequencies), and block or attenuate communications between the reader 124 and the at least one of the RFID tag 126A, 126B sent according to one or more other communications protocol(s) (e.g., protocols with relatively low power levels and/or relatively high carrier frequencies).

The rover controller 120 may be configured to operate the reader 124 according to both the blocked or attenuated communications protocols and the permitted communications protocols. Responsive to the reader 124 receiving a response from at least one of the RFID tags 126A, 126B when operating according to one of the blocked or attenuated communications protocols, and/or not receiving a response from at least one of the RFID tags 126A, 126B when operating according to one of the permitted communications protocols, the rover controller 120 may be configured to determine that an authentic manifold 116 is not inserted in the receiver 112. Conversely, responsive to the reader 124 not receiving a response from at least one of the RFID tags 126A, 126B when operating according to one of the blocked or attenuated communications protocols, and/or receiving a response from at least one of the RFID tags 126A, 126B when operating according to one of the permitted communications protocols, the rover controller 120 may be configured to determine that an authentic manifold 116 is inserted in the receiver 112.

For instance, the blocking media 138 may be configured to block or attenuate communications according to one or more communications protocols relative to one of the RFID tags 126, such as the secondary RFID tag 126B, and not interfere with communications according to such communication protocol(s) relative to the other RFID tag 126, such as the primary RFID tag 126A. In this case, the rover controller 120 may be configured to determine that an authentic manifold 116 is not seated in the receiver 112 responsive to occurrence of at least one of the following: the reader 124 receiving a response from the RFID tag 126B and/or not the RFID tag 126A when operating according to one of the blocked or attenuated communications protocols; and the rover controller 120 not receiving a response from one of the RFID tags 126A, 126B when operating according to one of the permitted communications protocols. Conversely, the rover controller 120 may be configured to determine that an authentic manifold 116 is seated in the receiver 112 responsive to occurrence of at least one of the following: the reader 124 receiving a response from the RFID tag 126A and/or not the RFID tag 126B when operating according to one of the blocked or attenuated communications protocols; and the rover controller 120 receiving a response from both of the RFID tags 126A, 126B when operating according to one of the permitted communications protocols.

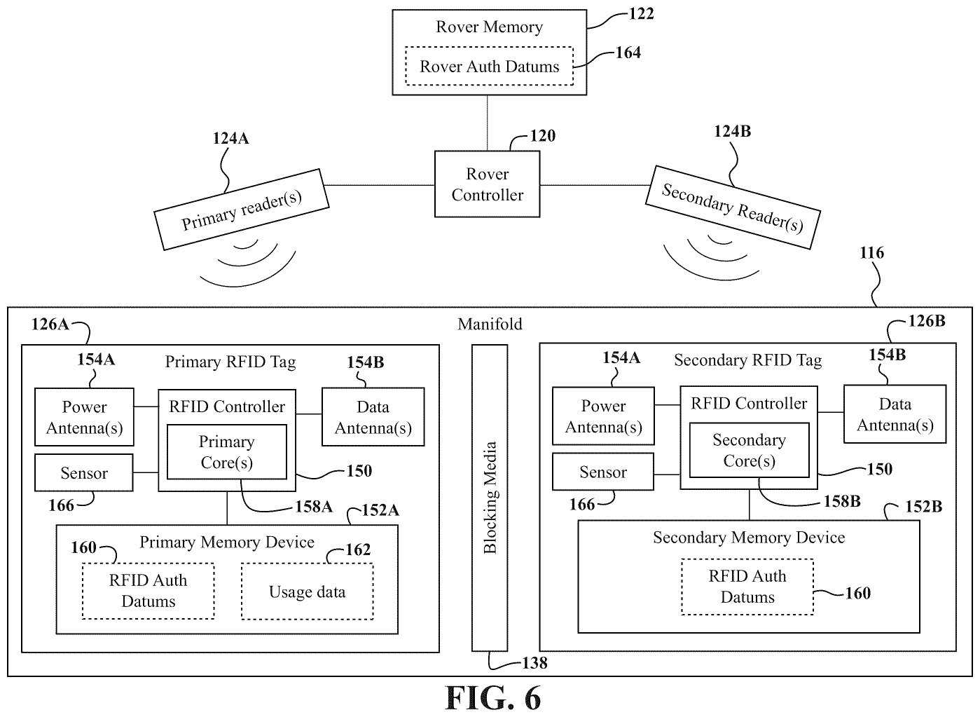

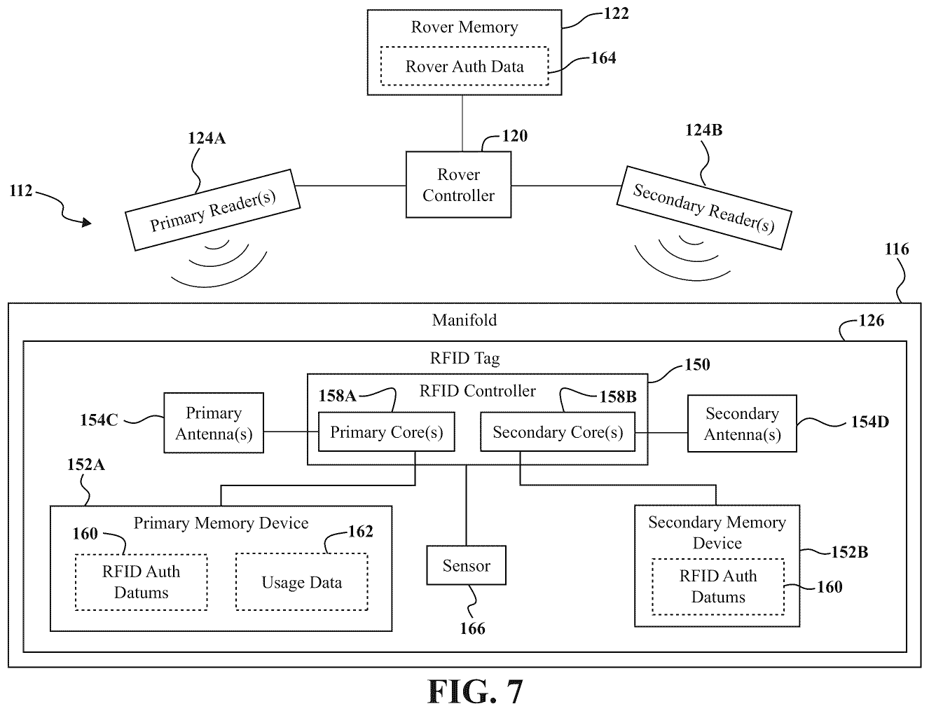

FIGS. 6 and 7 illustrate components that may be incorporated into each RFID tag 126 of a manifold 116. As shown in the illustrated examples, each RFID tag 126 may include an RFID controller 150, at least one memory device 152 in communication with the RFID controller 150, at least one antenna 154 coupled to the RFID controller 150, and a manipulation sensor 166. FIG. 6 specifically illustrates a manifold 116 including multiple RFID tags 126, namely a primary RFID tag 126A and a secondary RFID tag 126 including a primary memory device 152A and a secondary memory device 152B respectively. In some examples, the manifold 116 illustrated in FIG. 6 may also include a blocking media 138 disposed between the RFID tags 126, such as to inhibit certain communications from reaching the secondary RFID tag 126B as described above. FIG. 7 illustrates a manifold 116 having a single RFID tag 126 including multiple memory devices 152, namely a primary memory device 152A and a secondary memory device 152B. In some implementations, at least the memory devices 152 and RFID controller 150 of the RFID tag 126 of FIG. 7 may be incorporated on a same substrate and/or in a single integrated circuit.

The at least one antenna 154 of each RFID tag 126 may facilitate communication between the rover controller 120 and the RFID controller 150 via the one or more readers 124 of the rover 102. In some implementations, the RFID tag 126 may include multiple antennas 154 having different operating parameters, such as to enable communication between the RFID tag 126 and the rover controller 120 using different communications protocols. In other words, each antenna 154 may be configured to facilitate communication with a reader 124 via one or more different communications protocols. As some non-limiting examples, communication parameters varying across different communications protocols may include syntax, signal timing, signal current, signal power, signal voltage, carrier signal frequency, modulation type, and cryptography type, such as which cryptographic engine (e.g. cryptographic algorithm) is used for encrypting communications according to the communications protocol.

As illustrated in FIG. 6, in some implementations, each RFID tag 126 may include at least one power antenna 154A configured to harvest power for the RFID tag 126 from the reader(s) 124, and at least one data antenna 154B configured to receive and transmit data (e.g., authentication data) between the RFID tag 126 and the reader(s) 124, such according to one or more varying communications protocols and/or based on the data stored in the memory device(s) 152. Additionally or alternatively, as illustrated in FIG. 7, a given RFID tag 126 may include one or more primary antennas 154C for facilitating communication with the rover controller 120 relative to data stored in a primary memory device 152A of the RFID tag 126, and may include one or more secondary antennas 154D for facilitating communication with the rover controller 120 relative to data stored in a secondary memory device 152B of the RFID tag 126.

The primary antenna(s) 154C and the secondary antenna(s) 154D of a given RFID tag 126 may thus be respectively coupled to the primary memory device 152A and the secondary memory device 152B of the RFID tag 126, such as through the RFID controller 150. More specifically, the RFID controller 150 may be configured to process incoming communications and/or requests received through the primary antenna(s) 154C based on data stored in the primary memory device 152A, and may be configured to transmit communications corresponding to data stored in the primary memory device 152A via the primary antenna(s) 154C. Similarly, the RFID controller 150 may be configured to process incoming communications and/or requests received through the secondary antenna(s) 154D based on data stored in the secondary memory device 152B, and to transmit communications corresponding to data stored in the secondary memory device 152B via the secondary antenna(s) 154D. To this end, in some implementations, the RFID controller 150 may include one or more primary processing core(s) 158A optimized to process incoming communications and/or requests received through the primary antenna(s) 154C and/or corresponding to data residing in the primary memory device 152A, and may include one or more secondary processing core(s) 158B optimized to process incoming communications and/or requests received through the primary antenna(s) 154D and/or corresponding to data residing in the secondary memory device 152B.

As shown in the illustrated examples, a given receiver 112 of the rover 102 may include multiple readers 124 configured to communicate with the RFID tag(s) 126 of a manifold 116 when the manifold 116 is inserted into the receiver 112. In some implementations, each reader 124 may be configured to communicate with a different antenna 154 of the RFID tag 126, such as according to a different communications protocol. Referring to the illustrated examples, in some implementations, the receiver 112 may include at least one primary reader 124A configured to communicate with the primary antenna(s) 154C (FIG. 7) or the data antenna(s) 154B (FIG. 6) of each RFID tag 126, and may include at least one secondary reader 124B configured to communicate with the secondary antenna(s) 154D (FIG. 7) or the power antenna(s) 154A (FIG. 6) of each RFID tag 126. In some implementations, the readers 124 of each receiver 112 may be coupled to a same substrate and/or incorporated in a single integrated circuit.

The RFID controller 150 of each RFID tag 126 may generally be configured to implement the functions, features, and processes of the RFID tag 126 described herein. For instance, the RFID controller 150 may be configured to respond to interrogation signals received from the rover controller 120 of the rover 102, such as via the reader(s) 124 of the rover 102 and the antenna(s) 154 of the RFID tag 126. The RFID controller 150 may also be configured to execute commands received from the rover controller 120 relating to the memory device(s) 152 of the RFID tag 126, such as read and/or write commands, lock and/or unlock commands, lock status inquiry commands, and recommission commands.

To this end, the RFID controller 150 may include at least one processor configured to operate under control of computer-executable instructions residing in non-volatile storage, such as of the RFID controller 150 or of the memory device(s) 152 of the RFID tag 126. Specifically, the at least one processor may be configured to read the computer executable instructions into volatile storage (e.g., RAM) of the RFID controller 150 and then proceed to execute the same. As some non-limiting examples, the at least one processor may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital), such as based on operational instructions read from the non-volatile storage.

The computer-executable instructions executable by the RFID controller 150 may embody software programs, which may be compiled or interpreted from a variety of programming languages and/or technologies, including, without limitation, and either alone or in combination, Java, C, C++, C#, Objective C, Fortran, Pascal, Java Script, Python, Perl, and PL/SQL. For instance, the computer-executable instructions may embody one or more cryptography engines, each configured to execute a different cryptography algorithm, such as on authentication data received from the rover 102 (also referred to herein as “rover authentication data”) to authenticate the rover 102, or on data retrieved from a memory device 152 of the RFID tag 126 so as to generate authentication data to be communicated to the rover controller 120 to authenticate the manifold 116 (also referred to herein as “manifold authentication data”).

In some implementations, the RFID controller 150 of each RFID tag 126 may be a heterogeneous multicore device including a set of processing cores 158, or a set of primary processing cores 158A and/or a set of secondary processing cores 158B, with each processing core 158 of a given set being optimized to execute a different process or function of the RFID controller 150. For instance, each processing core 158 of a set may be optimized to execute a different cryptography engine. Correspondingly, the RFID controller 150 may be configured to periodically alternate cryptography engines, such as based on identification of a predefined event, and responsive to a given one of the cryptography engines becoming active, the RFID controller 150 may be configured to utilize the processing core 158 associated with the given cryptography engine to execute on data.

Each memory device 152 may include one or more forms of non-transitory computer-readable storage media, including volatile and non-volatile, and removable and nonremovable media, implemented for storage of information such as computer-readable instructions, data structures, program modules or other data. For example, each memory device 152 may include, without limitation, RAM, ROM, EPROM, EEPROM, flash memory device, or other solid state memory device technology.

In addition to computer-executable instructions, each memory device 152 may store data for determining whether a manifold 116 including the RFID tag 126 is usable with the rover 102. To this end, each memory device 152 may store one or more RFID authentication datums 160 for determining authenticity of the manifold 116. The authentication datums 160 may generally define one or more instances of authentication data for the manifold 116, such as one or more unique signatures for the manifold 116. Specifically, each RFID authentication datum 160 may indicate a parameter value to facilitate authenticating the manifold 116. For instance and without limitation, the authentication datum(s) 160 may include one or more signature datums, one or more hash digest datums, one or more key datums, one or more cryptographic engine datums, and/or one or more communications protocol datums.

In some implementations, the authentication datums 160 may form authentication datum sets each including a different combination of parameters for the different types of authentication datums 160 and/or defining a different instance of authentication data for the manifold 116. For instance, each signature and/or hash digest datum may be associated with a different key datum, a different cryptographic engine datum, and/or a different communications protocol datum within the memory device 252 so as to form the varying authentication datum sets. In some implementations, the RFID controller 150 may be configured to generate authentication data based on a given authentication datum set by, for example, applying the key and signature indicated by the authentication datum set to the cryptographic engine indicated by the authentication datum set to produce the authentication data, which may then be communicated by the RFID controller 150 to the rover controller 120 according to the communications protocol indicated by the authentication datum set. In alternative implementations, the RFID controller 150 may be configured to generate authentication data by, for example, applying a default encryption algorithm to a signature indicated by the one or more authentication datum(s) 160. The RFID controller 150 may be configured to activate and deactivate the varying authentication datums 160 or varying authentication datum sets for generating the authentication data as a function of various events, as discussed in more detail below.

At least one memory device 152 of the RFID tag(s) 126 of a given manifold 116, such as the primary memory device 152A, may also store usage data 162 indicative of usage of the manifold 116 to which the RFID tag(s) 126 is coupled. For instance, the usage data 162 may indicate whether suction has been provided through the manifold 116 and is thus used, a time since suction was initially provided through the manifold 116, and/or a volume of fluid collected through the manifold 116. The usage data 162 may also include information about the manifold 116 that may be used by the rover controller 120 to determine whether the manifold 116 may be operated with the rover 102. In some implementations, the secondary memory device 152B of the RFID tag(s) 126 of a given manifold 116 may include one or more RFID authentication datums 160 but omit usage data 162. In some instances, the secondary memory device 152B may be utilized for initial authentication of the manifold 116 and thereafter be rendered inaccessible as described in more detail below.

The manipulation sensor 166 of each RFID tag 126 may generally be configured to detect manipulation (e.g. reverse engineering efforts) of the RFID tag 126 and/or data stored on the memory device(s) 152 of the RFID tag 126. As some non-limiting examples, the manipulator sensor 166 may include one or more of a light sensor, a moisture sensor, or an electrical conductor configured to break if the RFID tag 126 is tampered. Upon receipt of an interrogation signal from the reader(s) 124, the RFID controller 150 may be configured to check the state of the manipulation sensor 166 to determine whether such state indicates tampering with the RFID tag 126. If so, then the RFID controller 150 may be configured to render the RFID tag 126 inoperable, and/or communicate a message to the rover controller 120 indicative of such determination, in which case the rover controller 120 may be configured to prevent activation of suction through the manifold 116, and indicate via the user interface 132 that the manifold 116 is not useable with the rover 102.

In general, the rover controller 120 and rover memory device 122 may be configured similar to the RFID controller(s) 150 and RFID memory device(s) 152 as described above. In other words, the rover controller 120 may include at least one processor configured to operate under control of computer-executable instructions residing in non-volatile storage, such as of the rover controller 120 or of the rover memory device 122. The rover memory device 122 may also store data facilitating operation of the rover controller 120 as described herein.

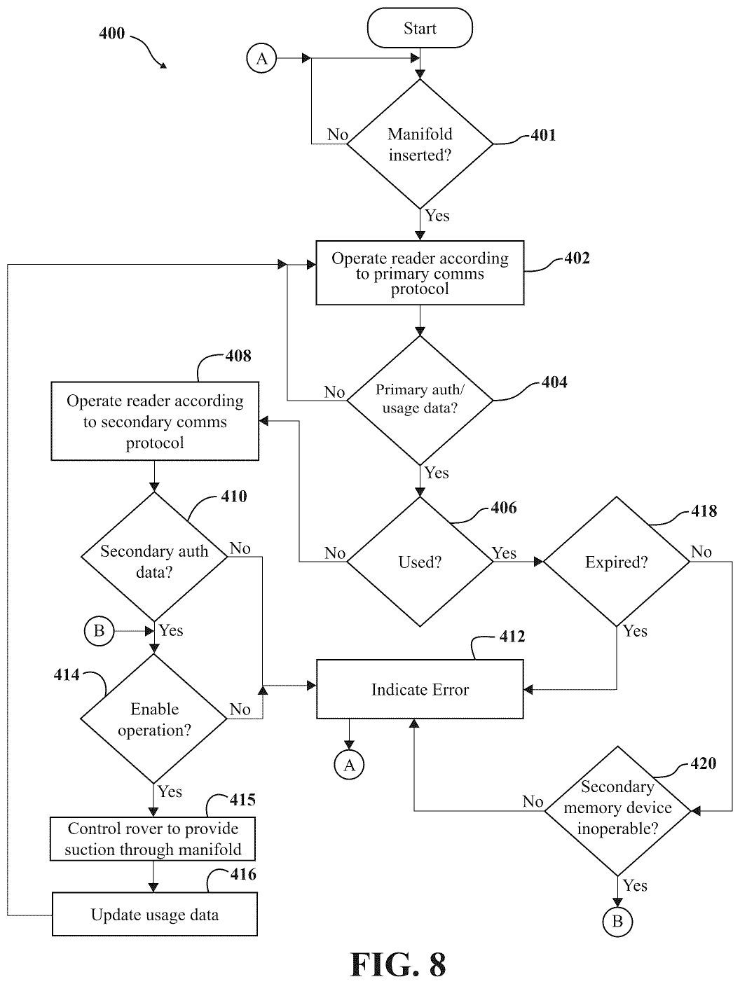

Referring now to FIG. 8, a flow diagram describing a process 400 for controlling the medical waste collection system 100 is illustrated. The process 400 may be carried out by the rover controller 120 via the reader(s) 124 of a given receiver 112, and may be used to authenticate a manifold 116 inserted into the receiver 112 based on authentication data received from the RFID tag(s) 126 of the manifold 116.

At 401, a determination may be made of whether a manifold 116 has been inserted into the receiver 112 of the rover 102. As an example, each receiver 112 may include a mechanical switch (e.g., switch 130 shown in phantom in FIG. 1) coupled to the rover controller 120, which may be configured to indicate when a manifold 116 is inserted into the receiver 112. Alternatively, the rover controller 120 may be configured to periodically operate the reader(s) 124 associated with each receiver 112 to emit a basic interrogation signal. Responsive to a reader 124 associated with the receiver 112 receiving a response from an RFID tag 126 to an interrogation signal following not receiving a response to an interrogation signal, the rover controller 120 may be configured to determine that a manifold 116 has been inserted into the receiver 112.

At 402, at least one of the reader(s) 124 associated with the receiver 112 may be operated according to a primary communications protocol, such as to elicit a response from the RFID tag(s) 126 that includes primary manifold authentication data and/or usage data 162 from the RFID tag(s) 126. The primary authentication data may correspond to at least one authentication datum 160 stored in a memory device 152 of the RFID tag(s) 126 in association with the primary communications protocol, such as at least one authentication datum 160 stored in a primary memory device 152A of the RFID tag(s) 126.

At 404, a determination may be made of whether the reader(s) 124 receive the primary authentication data and/or usage data 162 from the RFID tag(s) 126 responsive to operation of the reader(s) 124 according to the primary communications protocol. If such data is not received, the process 400 may return to 402 to attempt to elicit such data again. Otherwise, at 406, a determination may be made of whether the manifold 116 is used, such as based on the received usage data 162. The process 400 may then proceed along different paths depending on whether or not the manifold 116 is determined to be used.

If not, then at 408, at least one of the reader(s) 124 associated with the receiver 112 may be operated according to a secondary communications protocol, such as to elicit a response from the RFID tag(s) 126 that includes secondary authentication data. As an example, the secondary communications protocol may have a power level that is different (e.g., higher) than the power level of the primary communications protocol, and/or a carrier frequency that is different (e.g., lower) than the carrier frequency of the primary communications protocol. In general, the secondary authentication data may correspond to at least one authentication datum 160 stored in a memory device 152 of the RFID tag(s) 126 in association with the secondary communications protocol, such as at least one authentication datum 160 stored in a secondary memory device 152B of the RFID tag(s) 126. The secondary authentication data may thus differ from the primary authentication data.

At 410, a determination may be made of whether the reader(s) 124 receive the secondary authentication data from the RFID tag(s) 126 in response to the operation of the reader(s) 124 according to the secondary communications protocol. If the secondary authentication data is not received, then the process may proceed to 412 to indicate an error. For instance, the rover controller 120 may be configured to prevent activation of suction through the manifold 116, and indicate via the user interface 132 that the manifold 116 is not useable with the rover 102.

Conversely, if the secondary authentication data is received by the reader(s) 124 at 410, the process 400 may continue to 414. At 414, a determination may be made of whether to enable operation of the rover 102 to provide suction through the manifold 116, such as based on the first and second authentication data. As an example, the rover controller 120 may be configured to verify the first and second authentication data, and enable suction through the manifold 116 to be activated responsive to the authentication data being verified. In some implementations, the rover controller 120 may be configured to compare the first and second authentication data to a whitelist, which may be maintained on a remote system in communication with the rover 102, to verify the authentication data, and/or apply a verification algorithm to the authentication data. In some implementations, the rover controller 120 may also be configured to apply one or more cryptographic algorithms to the primary and secondary authentication data prior to verification, such as by applying one cryptographic algorithm to the primary authentication data and another cryptographic algorithm, different from the previous cryptographic algorithm, to the secondary authentication data. In some implementations, the selection by the rover controller 120 of the cryptographic algorithm to apply to each of the primary and secondary authentication data may be based on the RFID authentication datum 160 used to generate the authentication data, which as described herein may be a function of the communications protocol used to request the authentication data or occurrence of a predefined event.

Additionally or alternatively, such as following application of the cryptographic algorithms, the rover controller 120 may be configured to combine the (decrypted) primary and secondary authentication data to form an originality signature, which may then be compared to a whitelist or applied to a verification algorithm to verify the manifold 116. In some implementations, verification of received primary and secondary authentication data may be performed as described in Applicant's U.S. patent application Ser. No. 18/103,942, filed Jan. 31, 2023, the entire contents of which are hereby incorporated by reference herein in their entirety.

In some implementations, the primary authentication data read from the RFID tag(s) 126 may also include data to facilitate reading and/or verifying the secondary authentication data read from the RFID tag(s) 126. As an example, the RFID tag(s) 126 may be configured to communicate an indication of at least one RFID authentication datum 160 corresponding to the secondary authentication data, which as described above may indicate at least one of a communications protocol for requesting the secondary authentication data, an cryptographic algorithm for decrypting the secondary authentication data, a key for decrypting the secondary authentication data, and/or a hash digest for verifying the secondary authentication data. The rover controller 120 may thus utilize the received data to determine how to request and/or verify the secondary authentication data, which may differ from the manner in which the rover controller 120 requests and/or verifies the primary authentication data.

Still referring to FIG. 8, responsive to the manifold 116 not being authenticated for use with the rover 102 at 414, the process 400 may proceed to 412 to indicate an error as described above. Conversely, responsive to the manifold 116 being authenticated for use with the rover 102 at 414, the process may proceed to 415. At 415, operation of the rover 102 may be controlled to provide suction through manifold 116. For instance, the rover controller 120 may be configured to provide an indication via the user interface 132 that suction through the manifold 116 may be activated. Responsive to thereafter receiving a user input via the user interface 132 requesting activation of suction, the rover controller 120 may be configured to activate the vacuum pump 110 to provide the suction through the manifold 116.

At 416, the usage data 162 stored on the one or more RFID tag(s) 126 of the manifold 116 may be updated. As an example, the rover controller 120 may be configured to communicate the updated usage data 162 to the RFID tag(s) 126 through the reader(s) 124 as suction is applied through the manifold 116. For instance, the updated usage data 162 may include an indication that suction has been applied through the manifold 116, a time stamp corresponding to the first suction application, a volume of fluid that has passed through the manifold 116, and/or a number of insertions of the manifold 116 into a receiver 112.

The process 400 may end following 416. Alternatively, in some implementations and as shown in the illustrated example, following 416, the process 400 may return to 402 to again operate the reader(s) according to the primary communications protocol as described above, and so on to again check that the manifold 116 is suitable for operation with the rover 102. In this way, during operation of the rover 102 with a given manifold 116, the rover controller 120 may be configured to periodically check that the manifold 116 continues to be suitable for operation with the rover 102 by returning to 402.

Referring again to 406 of FIG. 8, responsive to determining that the manifold 116 is used, such as based on the usage data 162 read from the RFID tag(s) 126, the process 400 may proceed to 418 to determine whether the manifold 116 is expired. As examples, a given manifold 116 may be rated for use for up to a predefined time period from first insertion or first suction, and/or up to a predefined number of insertions, and/or up to a predefined volume of fluid. When the usage data 162 read from the RFID tag(s) 126 indicates the manifold 116 is used, the usage data 162 may be compared to one or more of the above-described thresholds to determine whether the manifold 116 is expired. Responsive to the comparison indicating that a value indicated by the usage data 162 equals or exceeds a corresponding threshold, the process 400 may consider the manifold 116 as expired and proceed to 412 to indicate an error as described above.

Assuming the manifold 116 is not determined to be expired, at 420, a determination may be made of whether a memory device 152 of the manifold 116 has been rendered inoperable. As described herein, a given manifold 116 may include multiple memory devices 152 incorporated therewith, each storing one or more authentication datums 160 for generating authentication data transmitted to the rover controller 120. For instance, a given manifold 116 may incorporate a single RFID tag 126 including a primary memory device 152A and a secondary memory device 152B, or may incorporate a primary RFID tag 126A and a secondary RFID tag 126B including a primary memory device 152A and a secondary memory device 152B respectively. Responsive to authentication data being generated based on data stored in a secondary memory device 152B or to suction being applied to the manifold 116, the secondary memory device 152B may be rendered inoperable. For instance, the rover controller 120 may be configured to transmit a kill command to the relevant the RFID tag 126, such as the secondary RFID tag 126B, that renders the RFID tag 126 or the secondary memory device 152B inoperable, or the RFID controller 150 of the implicated RFID tag 126 may be programmed to render itself or the secondary memory device 152B inoperable, such as upon transmission by the RFID controller 150 of authentication data based on an RFID authentication datum 160 stored in the secondary memory device 152B.

At 420, the rover controller 120 may thus be configured to determine whether the secondary memory device 152B of the manifold 116 has been rendered inoperable, such as by attempting to read and/or write data from and/or to the secondary memory device 152B, or by attempting to access the secondary RFID tag 126B including the secondary memory device 152B. Should the rover controller 120 be able to communicate with the secondary RFID tag 126B or the secondary memory device 152B, a determination may be made that the secondary memory device 152B is not inoperable. Correspondingly, the process 400 may move to 412 to indicate an error as described herein.

Conversely, should the rover controller 120 be unable to communicate with the secondary RFID tag 126B or the secondary memory device 152B, the process 400 may proceed to 414 in which a determination may be made whether to enable operation of the rover 102 to provide suction through the manifold 116, as described above. The determination in this case may be based on at least the previously received primary authentication data, such as described above, and/or without consideration of any secondary authentication data. Responsive to determining to enable operation of the rover 102 to provide suction through the manifold 116, at 415, operation of the rover 102 may be controlled to provide suction through the manifold 116, such as described above. At 416, usage data 162 of the RFID tag 126 may be updated as also described above, and so on.

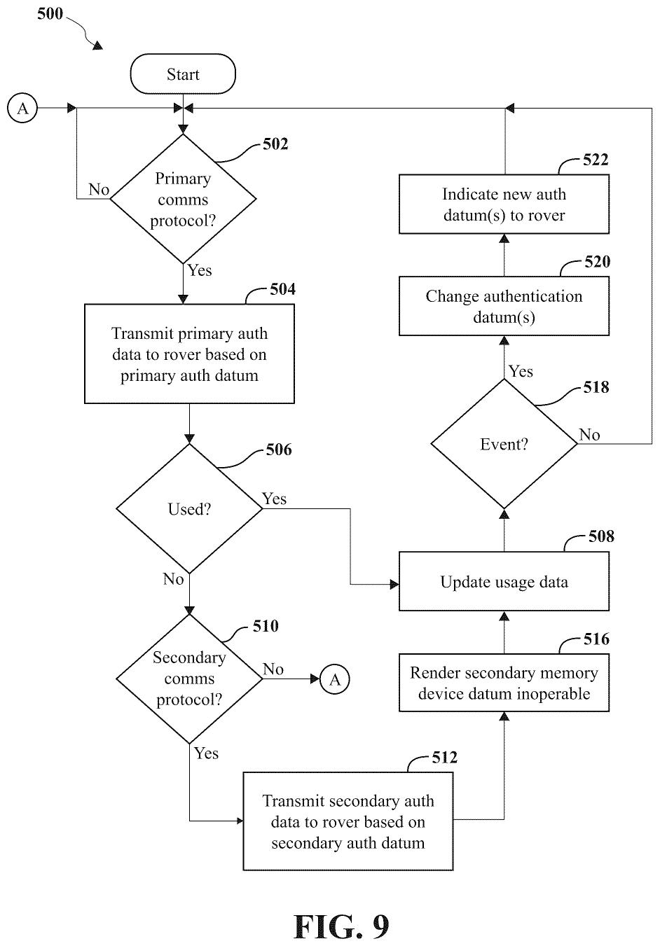

Referring to FIG. 9, a flow diagram illustrating a process 500 for operating one or more RFID tags 126 of a manifold 116 to facilitate authentication of the manifold 116 is provided. The process 500 may be carried out by the RFID controller(s) 150 of the RFID tag(s) 126 of the manifold 116, such as upon insertion of the manifold 116 into a receiver 112 of the rover 102.

At 502, a determination may be made of whether the RFID tag(s) 126 receive a communication according to a primary communications protocol, such as a communication including a request for primary authentication data of the manifold 116. If so, then at 504, primary authentication data may be transmitted to the rover 102, such as via the one or more antennas 154. More specifically, the RFID controller(s) 150 may be configured to generate and communicate the primary authentication data based on one or more of the authentication datum(s) 160 stored by the RFID tag(s) 126, or more specifically based on one or more authentication datum(s) 160 stored in the primary memory device 152A of the RFID tag(s) 126.

For instance, in some implementations, the stored authentication datum(s) 160 may indicate multiple parameter values each for generating different authentication data for the manifold 116, with each parameter value be associated with a different communications protocol. To this end, the RFID tag(s) 126 may be configured to generate and communicate varying authentication data as a function of the communications protocol in which requests for authentication data are received. In some implementations, the RFID tag(s) 126 may include antennas 154 each sensitive to a different communications protocol. In this way, the RFID controller(s) 150 may be configured to identify the communications protocol of a received request based on the antenna 154 in which the request is received.

As previously described, in some instances, the manifold 116 may include a primary RFID tag 126A and a secondary RFID tag 126 including a primary memory device 152A and a secondary memory device 152B respectively. Alternatively, the manifold 116 may include an RFID tag 126 including a primary memory device 152A and a secondary memory device 152B. In either case, in some implementations, each of the primary memory device 152A and the secondary memory device 152B may be associated with a different communications protocol, and may store one or more authentication datums 160 for generating authentication data transmitted to the rover controller 120. To this end, the RFID tag(s) 126 may include one or more antennas 154 coupled to the primary memory device 152A that are sensitive to the primary communications protocol but not the secondary communications protocol, and may include one or more antennas 154 coupled to the secondary memory device 152B that are sensitive to the secondary communications protocol but not the primary communications protocol. Correspondingly, responsive to the RFID tag(s) 126 receiving a request for authentication data via the antenna(s) 154 sensitive to the primary communications protocol, the RFID tag(s) 126 may be configured to generate and communicate to the rover controller 120 primary authentication data based on one or more of the authentication datum(s) 160 stored in the primary memory device 152A, and responsive to the RFID tag(s) 126 receiving a request for authentication data via the antenna(s) 154 sensitive to the secondary communications protocol, the RFID tag(s) 126 may be configured to generate and communicate to the rover controller 120 secondary authentication data based on one or more of the authentication datum(s) 160 stored in the secondary memory device 152B.

In some implementations, the communication received at 502 may also include rover authentication data, which may correspond to one or more rover authentication datums 164 stored in the rover memory device 122. The rover authentication datum(s) 164 may generally correspond to the RFID authentication datums 160 described above, and thus indicate similar data (e.g., parameter values for generating and/or communicating the rover authentication data). In this case, 504 may further include verifying the received rover authentication data, such as by applying a decryption and/or verification algorithm to the rover authentication data. For instance, the rover controller(s) 120 may be configured to apply a decryption algorithm to the rover authentication data to produce a first output indicative of a rover signature, and verify the rover signature against a whitelist or verification algorithm. Assuming the rover authentication data is verified, the rover controller 120 may be configured to generate and transmit the primary authentication data to the rover 102 according to one or more of the RFID authentication datums 160 as described herein.

In some implementations, the RFID controller(s) 150 may be configured to transmit the first output to the rover 102 along with the first authentication data. As an example, assuming the manifold 116 incorporates a primary RFID tag 126A and a secondary RFID tag 126B each storing authentication datum(s) 160, the RFID controller 150 of the primary RFID tag 126A may be configured, responsive to receiving an authentication data request according to the first communications protocol, to generate the first output and primary authentication data based on one or more of the authentication datum(s) 160 stored in the primary memory device 152A, and transmit such data to the rover controller 120 via the reader 124.

Alternatively, in other implementations, assuming the manifold 116 incorporates an RFID tag 126 including a primary memory device 152A and a secondary memory device 152B, responsive to receiving an authentication data request according to the first communications protocol, the RFID controller 150, or more particularly the primary processing core(s) 158A of the RFID controller 150, may be configured to generate and communicate the primary authentication data to the rover controller 120 based on the authentication datum(s) 160 stored in the primary memory device 152A, and generate and communicate the first output to the processing core(s) 158B associated with the secondary memory device 152B for further verification, as described in more detail below.

At 506, a determination may be made of whether the manifold 116 is used, such as based on the usage data 162 stored in the RFID tag(s) 126 incorporated with the manifold 116. If so, then the process 500 may proceed to 508 and update the usage data 162 based on corresponding data received from the rover controller 120, such as at 416 of the process 400 illustrated in FIG. 8.

Conversely, if it is determined that the manifold 116 is not used, then the process 500 may proceed to 510 to determine whether a further communication is received from the reader(s) 124 according to a secondary communications protocol that differs from the primary communications protocol. The further communication may include a second request for authentication data from the RFID tag(s) 126.

In some implementations, 510 may also include determining whether the communication according to the secondary communications protocol is received after termination of a relatively short predefined period and/or before the termination of a relatively long predefined period from receiving the communication according to the primary communications protocol and/or from transmitting the primary authentication data. The predefined periods may thus define a window following the communication according to the first communications protocol or the transmission of the primary authentication data in which to receive the communication according to the secondary communications protocol. If the communication according to the secondary communications protocol is received outside this window, or alternatively if the communication according to the secondary communications protocol is not received within this window, the process 500 may proceed back to 502 to again determine whether a communication according to the primary communications protocol is received, and so on.

Responsive to receiving the communication according to the secondary communications protocol at 510, at 512, secondary authentication data maybe generated and communicated from the RFID tag(s) 126 to the rover controller 120. As an example, assuming the manifold 116 incorporates multiple RFID tags 126, the RFID controller 150 of one of the RFID tags 126 that is different from the RFID tag 126 that generated and communicated the primary authentication data (e.g., the secondary RFID tag 126B) may be configured to generate and communicate the secondary authentication data based on one or more of the authentication datums 160 stored in the RFID tag 126 (e.g., in the secondary memory device 152B). As an alternative example, assuming the manifold 116 incorporates an RFID tag 126 including multiple memory devices 152 as described herein, the at least one processing core 158 of the RFID tag 126 that is different from the at least one processing core 158 that generated the primary authentication data (e.g., the secondary processing cores 158B) may be configured to generate and communicate the secondary authentication data, such as based on one of the authentication datums 160 stored in the memory device 152 to which the at least one processing core 158 is coupled (e.g., the secondary memory device 152B).

In some implementations, the communication received at 510 may also include rover authentication data, which may or may not differ from the rover authentication data communicated at 502. In some implementations, such as when the manifold 116 incorporates multiple RFID tags 126 disposed at different positions as described herein, the communication received at 510 may also include the first verification output previously generated and communicated to the rover controller 120. Alternatively, such as when the manifold 116 includes an RFID tag 126 having multiple memory devices 152, the first verification output may have been previously directly exchanged between the primary and secondary processing core(s) 158A, 158B, as described above.

The RFID controller 150 of the secondary RFID tag 126B, or the secondary processing core(s) 158B of the RFID tag 126, may be configured to generate and transmit the secondary authentication data based on the first output, the received rover authentication data, and at least one of the authentication datums 160 stored in the secondary memory device 152B. As an example, the RFID controller 150 of the secondary RFID tag 126B, or the secondary processing core(s) 158B, may be configured to execute a verification algorithm that takes as inputs the first output and the rover authentication data. Responsive to the verification algorithm outputting a positive verification, the RFID controller 150 of the secondary RFID tag 126B, or the secondary processing core(s) 158B of the RFID tag 126, may be configured to generate the secondary authentication data based on one or more of the authentication datums 160 stored in the secondary memory device 152B, and thereafter communicate the secondary authentication data to the rover controller 120 via the antenna(s) 154 and reader(s) 124.

At 516, the secondary memory device 152B may be rendered inoperable. As described above, the inoperability of the secondary memory device 152B may serve as a layer of authentication performed by the rover controller 120. In some implementations, the RFID controller 150 of the secondary RFID tag 126B and/or the secondary processing core(s) 158B may be configured to generate a kill signal that renders the secondary memory device 152B inaccessible, such as by overloading the RFID controller 150, the secondary processing core(s) 158B, and/or the secondary memory device 152B. The process 500 may then proceed to 508 to update the usage data 162 as described above.

At 518, a determination may be made of whether a predefined event has occurred. Occurrence of a predefined event may generally cause the RFID tag(s) 126 to select new authentication datum(s) 160 for the generation of authentication data. As some non-limiting examples, such predefined events may include a command from the reader(s) 124 to change authentication datum(s) 160, a passage of a predefined time period since selecting a given authentication datum 160 to generate authentication data, a communication timeout, such as after receiving a communication according to the primary communications protocol (e.g., not receiving the communication according to the secondary communications protocol within the predefined window), and/or a termination of a communication session between the reader(s) 124 and the RFID tag(s) 126, such as upon the RFID tag(s) 126 communicating the secondary authentication data to the reader(s) 124.

At 520, responsive to determining the occurrence of a predefined event, a different one or more of the authentication datums 160 may be selected for generating the authentication data as described herein. As an example, the RFID controller(s) 150, such as that of the primary RFID tag 126A, may be configured to select a different one or more of the authentication datums 160 of the primary memory device 152A for generating and/or communicating primary authentication data responsive to further primary authentication data requests. Similarly, assuming an implementation in which the secondary memory device 152B is not rendered inoperable (e.g., 516 is omitted from the from the process 500), the RFID controller(s) 150, such as that of the secondary RFID tag 126B, may be configured to select a different one or more of the authentication datums 160 of the secondary memory device 152B for generating and/or communicating secondary authentication data responsive to further secondary authentication data requests It will be appreciated that such activity may also function to change the primary and secondary communications protocols for requesting the primary and secondary authentication data, respectively. In other words, responsive to the RFID tag(s) 126 receiving a request for primary or secondary authentication data according to a communications protocol other than is currently active for the authentication data, the RFID controller(s) 150 may be configured to reject such request.

At 522, an indication of the newly selected authentication datums 160 may be communicated to the rover controller 120 via the reader(s) 124. Responsive to receiving such indication, the rover controller 120 may proceed to verify received authentication data according to the selected authentication datum(s) 160, such as described above. The process 500 may then return to 502 to determine whether a communication, such as an authentication data request, is again received via a primary communications protocol, and so on.

It is contemplated that in some implementations, not all the layers of authentication described in reference to FIGS. 8 and 9 may be performed. For instance, the verification that a memory device 152 has been rendered inoperable may be omitted. Additionally or alternatively, dual authentication as described above may be omitted, such that the RFID tag(s) 126 may not be configured to verify authentication data received from the rover 102. Additionally or alternatively, in some implementations, the rover controller 120 may be configured to permit operation of the rover 102 to provide suction through a manifold 116 based on one instance of authentication data received from the RFID tag(s) 126, and/or based on three more instances of received authentication data, each corresponding to a different one or more authentication datums 160.

It is also contemplated that verification of the received manifold authentication data (e.g., the primary authentication data and/or secondary authentication data) may be performed by a processing system remote from the rover 102, such as a cloud server or hub. More particularly, the rover controller 120 may be configured to communicate the manifold authentication data received from the RFID tag(s) 126 of an inserted manifold 116 to such remote processing system, such as over one or more private and/or public networks including the internet, and over one or more wired and/or wireless connections, for implementation of one or more of the authentication layers as described above. To this end, the rover 102 may further include a communications transceiver for establishing connections with such remote processing system. In some examples, the layers of authentication described in FIG. 8 may be split between the rover controller 120 and the remote processing system. For instance, the remote processing system may be configured to perform authentication of the manifold authentication data, and the rover controller 120 may be configured to determine if the manifold 116 is expired and/or includes an inoperable secondary memory device 152B.

In some implementations, the remote processing system may be a hub, such as a locally located hub (e.g., located in same facility as connected surgical devices), in communication with one or several surgical devices, such as several rovers 102, and such as via a local network (e.g., WiFi, Bluetooth). The hub may thus be configured to perform the authentication processes for each surgical device paired to the hub, such as based on data received from the surgical device (e.g., data read from a received manifold 116). In some instances, the hub may also be configured to pull data from a remote server to facilitate the authentication processes, such as updated keys, updated predefined lock patterns, and updated authentication algorithms corresponding to a given received manifold 116. As one example, the hub may be similar to that described in U.S. Patent Publication No. 2022/0317827, published Oct. 6, 2022, which is hereby incorporated by reference herein in its entirety. In some implementations, the hub may also be configured to forward such updates to the rover(s) 102 to enable local authentication by the rover(s) 102 based thereon.

In some instances, the above-described hub may be incorporated into a docking station for the rover 102. The docking station may be configured to receive the rover 102 for emptying and cleaning the container(s) 108. As one example, the docking station may be similar to that described in International Publication No. WO 2007/070570, published Jun. 21, 2007, which is hereby incorporated by reference herein in its entirety.

In some instances, such as when the rover controller 120 is configured to perform the aforementioned authentication locally, communication between the docking station and rover 102 may be limited to proximity- or line of site-based connections (e.g., infrared, NFC, or RFID), which may be established when the rover 102 is docked with the docking station. In this case, the docking station may be configured to periodically receive any updated keys, predefined lock patterns, and authentications algorithms from a remote server, and communicate such updated information to the rover 102 for the authentication of manifolds 116 when the rover 102 is next docked to the docking station.

It is contemplated that each of the above-described RFID tags 126 may be replaced with a suitable alternative device, such as an alternative type of tag, memory device, or a controller including a memory device, that is communicatively coupled with the rover controller 120 upon the manifold 116 being received in the rover 102 for the exchange of data and/or authentication as described above. For instance, the manifold 116 may include at least one controller and/or memory device configured to establish a wired data connection, or alternatively an IR data connection, with the rover controller 120 upon the manifold 116 being received. As a further example, the manifold 116 may include at least one barcode encoded with the above-described data, with the rover controller 120 being coupled to at least one barcode reader such that the rover controller 120 is able to read the barcode(s) upon the manifold 116 being received by the rover 102.

Although described above in the context of a manifold 116 for a rover 102, a controller and/or memory device and/or tag described above may be used with a variety of medical/surgical devices and/or systems. For example, the following devices are contemplated: 1) Lighting devices comprising a controller and/or memory device and/or tag, such as the lighting device described in U.S. Pat. No. 10,226,555, which is hereby incorporated by reference herein in its entirety; 2) Suction devices comprising a controller and/or memory device and/or tag, such as the suction devices described in U.S. Pat. Nos. 11,376,093, 9,510,737 and/or 10,499,974, which are hereby incorporated by reference herein in their entirety; 3) Surgical garments comprising a controller and/or memory device and/or tag, such as the surgical garments described in U.S. Pat. Nos. 11,090,516 and/or 11,291,265, which are hereby incorporated by reference herein in their entirety; 4) Electrosurgical devices, such as electrosurgical pencils or forceps, comprising a controller and/or memory device and/or tag, such as the electrosurgical devices described in U.S. Pat. Nos. 10,70,912 and/or 8,361,070, which are hereby incorporated by reference herein in their entirety; 5) Retractors comprising a controller and/or memory device and/or tag, such as the retractors described in U.S. Pat. Nos. 11,351,004 and/or 11,382,711, which are hereby incorporated by reference herein in their entirety; 6) Smoke filters comprising a controller and/or memory device and/or tag, such as the smoke filters described in U.S. Pat. Nos. 11,160,909 and/or 7,761,188, which are hereby incorporated by reference herein in their entirety; 7) Irrigation sleeves for ultrasonic devices comprising a controller and/or memory device and/or tag, such as the irrigation sleeve described in U.S. Pat. No. 11,317,936, which is hereby incorporated by reference herein in its entirety; 8) Irrigation cassettes for surgical consoles comprising a controller and/or memory device and/or tag, such as the cassettes described in U.S. Pat. Nos. 7,632,079 and/or 8,35,487, which are hereby incorporated by reference herein in their entirety; 9) Intrauterine devices comprising a controller and/or memory device and/or tag, such as the intrauterine devices described in U.S. Provisional App. No. 63/323,677 and/or PCT/US22/41636, which are hereby incorporated by reference herein in their entirety; 10) Surgical sponges or other surgical objects comprising a controller and/or memory device and/or tag, such as the surgical sponges described in U.S. Pat. Nos. 7,703,674 and/or 11,116,598, which are hereby incorporated by reference herein in their entirety; and 11) Sterilization trays comprising a controller and/or memory device and/or tag, such as the sterilization tray described in 5,540,901, which is hereby incorporated by reference herein in its entirety.

Methods for preparing an RFID tag 126 (e.g., obtaining and programming an RFID tag) and for preparing a manifold 116 (e.g., preparing RFID tag and coupling to manifold) in accordance with and to facilitate the above exemplary implementations are also contemplated. In addition, it is contemplated that such methods may be applied to controllers and/or memory devices and/or tags for the above alternative devices. Furthermore, it is contemplated that the rover 102 may be replaced by an appropriate alternative device, such as a surgical console, a surgical handpiece, a surgical helmet, a smoke filtration console, a sponge scanner, etc., and hence, these devices could perform the methods described herein with respect to the rover 102 and/or rover controller 120. It is also contemplated that a controller and/or memory device and/or tag described above may be incorporated into a cord or connector of a surgical device or instrument, such as an electrical cord or connector of a handheld surgical device or instrument (e.g., the cord connector of an electrosurgical or ultrasonic surgical instrument). In this way, a data connection may be formed between the controller and/or memory device and/or tag upon the connector of the surgical device or instrument being coupled to a receiving device, such as a surgical console, which may in turn be configured to perform the authentication routine(s) described above.

In general, the routines executed to implement aspects of the disclosure, whether implemented as part of an operating system or a specific application, component, program, object, module or sequence of instructions, or even a subset thereof, may be referred to herein as “computer program code,” or simply “program code.” Program code may comprise computer readable instructions that are resident at various times in various memory device and storage devices in a computer and that, when read and executed by one or more processors in a computer, cause that computer to perform the operations necessary to execute operations and/or elements embodying the various aspects of the disclosure. Computer readable program instructions for carrying out operations of the various aspects of the disclosure may be, for example, assembly language or either source code or object code written in any combination of one or more programming languages.

The program code embodied in any of the applications/modules described herein may be capable of being individually or collectively distributed as a program product in a variety of different forms. In particular, the program code may be distributed using a computer readable storage medium having computer readable program instructions thereon for causing a processor to carry out aspects of the present disclosure.

Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include random access memory device (RAM), read-only memory device (ROM), erasable programmable read-only memory device (EPROM), electrically erasable programmable read-only memory device (EEPROM), flash memory device or other solid state memory device technology, portable compact disc read-only memory device (CD-ROM), or other optical storage, magnetic cassettes, magnetic tape, magnetic disk storage or other magnetic storage devices, or any other medium that can be used to store the desired information and which can be read by a computer. A computer readable storage medium should not be construed as transitory signals per se (e.g., radio waves or other propagating electromagnetic waves, electromagnetic waves propagating through a transmission media such as a waveguide, or electrical signals transmitted through a wire). Computer readable program instructions may be downloaded to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions/acts specified in the flowcharts, sequence diagrams, and/or block diagrams. The computer program instructions may be provided to one or more processors such that the instructions, which execute via the one or more processors, cause a series of computations to be performed to implement the functions and/or acts specified in the flowcharts, sequence diagrams, and/or block diagrams described herein.

In certain alternatives, the functions and/or acts specified in the flowcharts, sequence diagrams, and/or block diagrams may be re-ordered, processed serially, and/or processed concurrently without departing from the scope of the disclosure. Moreover, any of the flowcharts, sequence diagrams, and/or block diagrams may include more or fewer blocks than those illustrated herein.

The terminology used herein is for the purpose of describing particular examples only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “includes,” “having,” “has,” “with,” “comprised of,” or variants thereof are used in either the detailed description or the claims, such terms are intended to be inclusive in a manner similar to the term “comprising.”

While this description includes various examples and while these examples have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

Some examples are described with reference to the following numbered clauses, with specific features laid out in dependent clauses:

Clause 1. A medical waste collection system comprising: a medical waste collection device for providing suction at a surgical site; and a manifold couplable to the medical waste collection device, the manifold defining a pathway through which the medical waste collection device is configured to provide the suction to the surgical site, the manifold including at least one RFID tag comprising a first memory device and a second memory device, the first and second memory devices storing first and second manifold authentication data respectively, wherein the medical waste collection device comprises: a waste container for collecting medical waste material; a receiver in fluid communication with the waste container and dimensioned to removably receive the manifold; at least one reader disposed adjacent the receiver and configured to communicate with the at least one RFID tag according to a first communications protocol and a second communications protocol when the manifold is inserted in the receiver; and at least one controller coupled to the at least one reader and configured to: operate the at least one reader according to the first communications protocol to read the first manifold authentication data from the first memory device of the at least one RFID tag, operate the at least one reader according to the second communications protocol to read the second manifold authentication data from the second memory device of the at least one RFID tag, and control operation of the medical waste collection device to provide suction based on the read first and second manifold authentication data.

Clause 2. The medical waste collection system of clause 1, wherein the first communications protocol differs from the second communications protocol in at least one of syntax, timing, power level, voltage level, current level, carrier frequency, modulation type, and data encryption algorithm.

Clause 3. The medical waste collection system of clause 1 or 2, wherein the first communications protocol has at least one of a higher carrier frequency and a lower power level than the second communications protocol.

Clause 4. The medical waste collection system of any one of clauses 1-3, wherein the at least one RFID tag comprises a first RFID tag including the first memory device and a second RFID tag including the second memory device, the first and second RFID tags being disposed at different positions of the manifold.

Clause 5. The medical waste collection system of clause 4, wherein the manifold comprises a proximal region and a distal region, the proximal region including a port for engaging a vacuum inlet integral with the medical waste collection device when the manifold is inserted in the receiver, and the distal region including a plurality of inlet fittings for engaging suction tubes extending to the surgical site, and wherein the first RFID tag is disposed along an outer surface of the manifold extending from the proximal region to the distal region, and the second RFID tag is disposed at the proximal region adjacent the port.

Clause 6. The medical waste collection system of clause 4 or 5, wherein the at least one reader comprises a first reader positioned for reading the first manifold authentication data from the first RFID tag when the manifold is inserted in the receiver, and comprises a second reader positioned for reading the second manifold authentication data from the second RFID tag when the manifold is inserted in the receiver.

Clause 7. The medical waste collection system of any one of clauses 4-6, wherein the manifold or medical waste collection device comprises a blocking media disposed adjacent the second RFID tag or the at least one reader respectively for preventing the at least one reader from communicating with the second RFID tag according to the first communications protocol.

Clause 8. The medical waste collection system of any one of clauses 1-3, wherein the first memory device and second memory device are incorporated in a single RFID tag.

Clause 9. The medical waste collection system of clause 8, wherein the single RFID tag includes a first antenna for communication via the first communications protocol and a second antenna for communication via the second communications protocol.

Clause 10. The medical waste collection system of clause 8, wherein the single RFID tag includes a first antenna for receiving a power signal from the at least one reader and a second antenna for communicating the first and second manifold authentication data to the at least one reader.

Clause 11. The medical waste collection system of any one of clauses 1-6 and 8-10, wherein the at least one reader is defined as only one reader.

Clause 12. The medical waste collection system of any one of clauses 1-11, wherein the at least one controller is configured to communicate a first request for the first manifold authentication data according to the first communications protocol to the at least one RFID tag, the first request including first rover authentication data, and the at least one RFID tag is configured to communicate the first manifold authentication data to the at least one reader based on the first rover authentication data.

Clause 13. The medical waste collection system of clause 12, wherein the at least one controller is configured to communicate a second request for the second manifold authentication data according to the second communications protocol to the at least one RFID tag, the second request including second rover authentication data that is different from the first rover authentication data, and the at least one RFID tag is configured to communicate the second manifold authentication data to the at least one reader based on the second rover authentication data.

Clause 14. The medical waste collection system of clause 13, wherein the at least one RFID tag is configured to: apply a first cryptographic algorithm to the first rover authentication data to produce a first output; communicate the first manifold authentication data to the at least one reader based on the first output; apply a second cryptographic algorithm, different from the first cryptographic algorithm, to the second rover authentication data to produce a second output; and communicate the second manifold authentication data to the at least one reader based on the second output.

Clause 15. The medical waste collection system of clause 13 or 14, wherein the at least one controller is configured to communicate the second request for the second manifold authentication data to the at least one RFID tag based on the first manifold authentication data received from the RFID tag.

Clause 16. The medical waste collection system of clause 15, wherein the first manifold authentication data indicates at least one authentication datum for the second request for the second manifold authentication data, the at least one authentication datum indicating at least one of an encryption key for generating the second request, an encryption algorithm for generating the second request, and the second communications protocol for communicating the second request.

Clause 17. The medical waste collection system of any one of clauses 13-16, wherein the at least one RFID tag comprises a first processing core and a second processing core, the first processing core being configured to: communicate the first manifold authentication data to the at least one reader responsive to the at least one RFID tag receiving the first request for the first manifold authentication data that includes the first rover authentication data; and communicate third manifold authentication data to the second processing core based on the first rover authentication data received from the at least one reader, wherein the second processing core is configured to communicate the second manifold authentication data to the at least one reader based on the second rover authentication data from the at least one reader and the third manifold authentication data from the first processing core.

Clause 18. The medical waste collection system of clause 17, wherein the third manifold authentication data indicates at least one authentication datum for verifying the second rover authentication data.

Clause 19. The medical waste collection system of any one of clauses 1-18, wherein the at least one controller is configured to: responsive reading the second manifold authentication data, determine whether the second memory device is inoperable using the at least one reader; and control operation of the medical waste collection device to provide suction based on the determination of whether the second memory device is inoperable.

Clause 20. The medical waste collection system of any one of clauses 1-19, wherein the at least one controller is configured to: apply a first cryptographic algorithm and a second cryptographic algorithm, different from the first cryptographic algorithm, to the read first and second manifold authentication data respectively to provide first and second outputs respectively; and control operation of the medical waste collection device to provide suction based on the first and second outputs.

Clause 21. The medical waste collection system of any one of clauses 1-20, wherein the at least one controller is configured to: combine the first and second manifold authentication data to form an originality signature, and control operation of the medical waste collection device to provide suction based on the combined originality signature.

Clause 22. The medical waste collection system of any one of clauses 1-21, wherein the at least one RFID tag is configured to, responsive to receiving a request according to the second communications protocol for the first manifold authentication data, reject the request.

Clause 23. A medical waste collection system comprising: a medical waste collection device for providing suction at a surgical site; and a manifold couplable to a medical waste collection device, the manifold defining a pathway through which the medical waste collection device is configured to provide the suction to the surgical site, the manifold including at least one RFID tag comprising a first memory device and a second memory device, the first and second memory devices storing first and second manifold authentication data respectively, wherein the medical waste collection device comprises: a waste container for collecting medical waste material; a receiver in fluid communication with the waste container and dimensioned to removably receive the manifold; at least one reader disposed adjacent the receiver and configured to communicate with the at least one RFID tag when the manifold is inserted in the receiver; and at least one controller coupled to the at least one reader and configured to: operate the at least one reader to read the first manifold authentication data from the first memory device of the at least one RFID tag, operate the at least one reader to read the second manifold authentication data from the second memory device of the at least one RFID tag, combine the first and second manifold authentication data to form an originality signature, and control operation of the medical waste collection device to provide suction based on the combined originality signature.

Clause 24. The medical waste collection system of clause 23, wherein the at least one RFID tag comprises a first RFID tag including the first memory device and a second RFID tag including the second memory device, the first and second RFID tags being located at different positions of the manifold.

Clause 25. The medical waste collection system of clause 24, wherein the manifold comprises a proximal region and a distal region, the proximal region including a port for engaging a vacuum inlet integral with the medical waste collection device when the manifold is inserted in the receiver, and the distal region including a plurality of inlet fittings for engaging suction tubes extending to the surgical site, and wherein the first RFID tag is disposed along an outer surface of the manifold extending from the proximal region to the distal region, and the second RFID tag is disposed at the proximal region adjacent the port.

Clause 26. The medical waste collection system of clause 24 or 25, wherein the at least one reader comprises a first reader positioned for reading the first manifold authentication data from the first RFID tag when the manifold is inserted in the receiver, and comprises a second reader positioned for reading the second manifold authentication data from the second RFID tag when the manifold is inserted in the receiver.

Clause 27. The medical waste collection system of clause 23 or 24, wherein the first memory device and second memory device are incorporated in a single RFID tag.

Clause 28. The medical waste collection system of clause 27, wherein the single RFID tag includes a first antenna for communication of the first manifold authentication data and a second antenna for communication of the second manifold authentication data.

Clause 29. The medical waste collection system of clause 27, wherein the single RFID tag includes a first antenna for receiving a power signal from the at least one reader and a second antenna for communicating the first and second manifold authentication data to the at least one reader.

Clause 30. The medical waste collection system of any one of clauses 23-25 and 27-29, wherein the at least one reader is defined as only one reader.

Clause 31. The medical waste collection system of any one of clauses 23-30, wherein the at least one controller is configured to communicate a first request for the first manifold authentication data to the at least one RFID tag, the first request including first rover authentication data, and the at least one RFID tag is configured to communicate the first manifold authentication data to the at least one reader based on the first rover authentication data.

Clause 32. The medical waste collection system of clause 31, wherein the at least one controller is configured to communicate a second request for the second manifold authentication data to the at least one RFID tag, the second request including second rover authentication data that is different from the first rover authentication data, and the at least one RFID tag is configured to communicate the second manifold authentication data to the at least one reader based on the second rover authentication data.

Clause 33. The medical waste collection system of clause 32, wherein the at least one RFID tag is configured to: apply a first cryptographic algorithm to the first rover authentication data to produce a first output; communicate the first manifold authentication data to the at least one reader based on the first output; apply a second cryptographic algorithm, different from the first cryptographic algorithm, to the second rover authentication data to produce a second output; and communicate the second manifold authentication data to the at least one reader based on the second output.

Clause 34. The medical waste collection system of clause 32 or 33, wherein the at least one controller is configured to communicate the second request for the second manifold authentication data to the at least one RFID tag based on the first manifold authentication data received from the RFID tag.

Clause 35. The medical waste collection system of clause 34, wherein the first manifold authentication data indicates at least one authentication datum for the second request for the second manifold authentication data, the at least one authentication datum indicating at least one of an encryption key for generating the second request, an encryption algorithm for generating the second request, and a communications protocol for communicating the second request.

Clause 36. The medical waste collection system of any one of clauses 32-35, wherein the at least one RFID tag comprises a first processing core and a second processing core, the first processing core being configured to: communicate the first manifold authentication data to the at least one reader responsive to the at least one RFID tag receiving the first request for the first manifold authentication data that includes the first rover authentication data; and communicate third manifold authentication data to the second processing core based on the first rover authentication data received from the at least one reader, wherein the second processing core is configured to communicate the second manifold authentication data to the at least one reader based on the second rover authentication data from the at least one reader and the third manifold authentication data from the first processing core.

Clause 37. The medical waste collection system of clause 36, wherein the third manifold authentication data indicates at least one authentication datum for verifying the second rover authentication data.

Clause 38. The medical waste collection system of any one of clauses 23-37, wherein the at least one controller is configured to: responsive reading the second manifold authentication data, determine whether the second memory device is inoperable using the at least one reader; and control operation of the medical waste collection device to provide suction based on the determination of whether the second memory device is inoperable.

Clause 39. The medical waste collection system of any one of clauses 23-28, wherein the at least one controller is configured to: apply a first cryptographic algorithm and a second cryptographic algorithm, different from the first cryptographic algorithm, to the read first and second manifold authentication data respectively to provide first and second outputs respectively; and control operation of the medical waste collection device to provide suction based on the first and second outputs.

Clause 40. A medical waste collection system comprising: a medical waste collection device for providing suction at a surgical site; and a manifold couplable to the medical waste collection device, the manifold defining a pathway through which the medical waste collection device is configured to provide the suction to the surgical site, the manifold including an RFID tag, wherein the medical waste collection device comprises: a waste container for collecting medical waste material; a receiver in fluid communication with the waste container and dimensioned to removably receive the manifold; at least one reader disposed adjacent the receiver and configured to communicate with the RFID tag when the manifold is inserted in the receiver; and at least one controller configured to operate the at least one reader to obtain authentication data from the RFID tag and to control operation of the medical waste collection device to provide suction based on the obtained authentication data, wherein the RFID tag is configured to: identify operation of the at least one reader according to a first communications protocol; transmit first authentication data to the at least one reader based on the identification of the first communications protocol; identify operation of the at least one reader according to a second communications protocol; and transmit second authentication data, different from the first authentication data, to the at least one reader based on the identification of the second communications protocol.

Clause 41. The medical waste collection system of clause 40, wherein the first communications protocol differs from the second communications protocol in at least one of syntax, timing, power level, voltage level, current level, carrier frequency, modulation type, and data encryption type.

Clause 42. The medical waste collection system of clause 40 or 41, wherein the at least one reader comprises a first reader configured to communicate with the RFID tag according to the first communications protocol and a second reader configured to communicate with the RFID tag according to the second communications protocol.

Clause 43. The medical waste collection system of any one of clauses 40-42, wherein the RFID tag includes a first antenna for communication via the first communications protocol and a second antenna for communication via the second communications protocol.

Clause 44. The medical waste collection system of any one of clauses 40-43, wherein the RFID tag includes a first antenna for receiving a power signal from the at least one reader and a second antenna for communicating the first and second authentication data to the at least one reader.

Clause 45. The medical waste collection system of any one of clauses 40-44, wherein the RFID tag is configured to: determine whether operation of the at least one reader according to the second communications protocol begins within a predefined window following the identification of the at least one reader being operated according to the first communications protocol; and transmit the second authentication data to the at least one reader responsive to determining that the operation of the at least one reader according to the second communications protocol begins within the predefined window.

Clause 46. The medical waste collection system of any one of clauses 40-45, wherein the RFID tag is configured to: receive third authentication data from the at least one reader being operated according to the first communications protocol; apply a cryptography algorithm to the third authentication data to produce a first output; and communicate the first authentication data to the at least one reader based on the first output.

Clause 47. The medical waste collection system of any one of clauses 40-46, wherein the RFID tag comprises first and second RFID processor cores configured to generate the first and second authentication data respectively.

Clause 48. The medical waste collection system of clause 47, wherein the first RFID processor core is configured to: receive third authentication data from the at least one reader being operating according to the first communications protocol; apply a first cryptography algorithm to the third authentication data to produce a first output; and communicate the first authentication data to the at least one reader based on the first output, and wherein the second RFID processor core is configured to: receive the third authentication data from the at least one reader being operated according to the second communications protocol; apply a second cryptography algorithm different from the first cryptography algorithm to the third authentication data to produce a second output; and communicate the second authentication data to the at least one reader based on the second output.

Clause 49. The medical waste collection system of any one of clauses 40-48, wherein the first authentication data indicates the second communications protocol.

Clause 50. The medical waste collection system of any one of clauses 40-49, wherein the at least one controller or the RFID tag is configured to render the first communications protocol inoperable for obtaining authentication data from the RFID tag responsive to identifying operation of the at least one reader according to the second communications protocol.

Clause 51. The medical waste collection system of clause 50, wherein the at least one controller is configured to: responsive to receiving the second authentication data, determine whether the first communications protocol is inoperable for obtaining authentication data from the RFID tag; and control operation of the medical waste collection device to provide suction based on the determination of whether the first communications protocol is inoperable.

Clause 52. The medical waste collection system of any one of clauses 40-51, wherein the RFID tag stores a plurality of authentication datums each associated with a different communications protocol, and is configured to: select a first of the authentication datums based on the identification of the first communications protocol and transmit the first authentication data to the at least one reader based on the first authentication datum; and select a second of the authentication datums based on the identification of the second communications protocol and transmit the second authentication data to the at least one reader based on the second authentication datum.

Clause 53. The medical waste collection system of any one of clauses 40-52, wherein each of the authentication datums indicates at least one of a unique cryptographic engine and a unique key.

Clause 54. The medical waste collection system of clause 52 or 53, wherein the RFID tag comprises a plurality of processing cores, and each of the authentication datums is associated with a different processor core configured to be made active when the authentication datum is selected.

Clause 55. The medical waste collection system of any one of clauses 40-54, wherein the at least one controller is configured to: apply a first cryptographic algorithm and a second cryptographic algorithm, different from the first cryptographic algorithm, to the first and second authentication data respectively to provide first and second outputs respectively; and control operation of the medical waste collection device to provide suction based on the first and second outputs.

Clause 56. The medical waste collection system of any one of clauses 40-55, wherein the at least one controller is configured to: combine the first and second authentication data to form an originality signature, and control operation of the medical waste collection device to provide suction based on the combined originality signature.

Clause 57. A medical waste collection system comprising: a medical waste collection device for providing suction at a surgical site; and a manifold couplable to the medical waste collection device, the manifold defining a pathway through which the medical waste collection device is configured to provide the suction to the surgical site, the manifold including an RFID tag, wherein the medical waste collection device comprises: a waste container for collecting medical waste material; a receiver in fluid communication with the waste container and dimensioned to removably receive the manifold; at least one reader disposed adjacent the receiver and configured to communicate with the RFID tag when the manifold is inserted in the receiver; and at least one controller configured to operate the at least one reader to obtain authentication data from the RFID tag and to control operation of the medical waste collection device to provide suction based on the obtained authentication data, wherein the RFID tag is configured to: responsive to receiving a first request from the at least one reader, communicate first authentication data to the at least one reader, the first authentication data being generated based on a first authentication datum; identify occurrence of a predefined event; and responsive to receiving a second request from the at least one reader after the predefined event, communicate second authentication data to the at least one reader, the second authentication data being generated based on a second authentication datum different from the first authentication datum.

Clause 58. The medical waste collection system of clause 57, wherein the RFID tag is configured to determine the second authentication datum based on the first authentication datum and responsive to the predefined event.

Clause 59. The medical waste collection system of clause 58, wherein the second authentication datum is a key mathematically related to the first authentication datum.

Clause 60. The medical waste collection system of clause 57 or 58, wherein the RFID tag includes a memory storing a plurality of authentication datums including the first and second authentication datums.

Clause 61. The medical waste collection system of clause 60, wherein each of the authentication datums indicates at least one of a unique cryptographic engine, a unique communications protocol, and a unique key.

Clause 62. The medical waste collection system of clause 60 or 61, wherein the RFID tag comprises a plurality of processor cores, each of the authentication datums being associated with a different one of the processor cores.

Clause 63. The medical waste collection system of clause 62, wherein each of the processor cores is configured to: receive third authentication data from the at least one reader; and process the third authentication data according to the authentication datum associated with the processing core to produce unique authentication data communicated to the at least one reader.

Clause 64. The medical waste collection system of any one of clauses 57-63, wherein the RFID tag is configured to, responsive to identifying the occurrence of the predefined event, communicate an indication to the at least one reader that the second authentication datum is active.

Clause 65. The medical waste collection system of any one of clauses 57-64, wherein the predefined event comprises at least one of passage of a predefined time period from receiving the first request, a communication timeout, a termination of a communication session between the at least one reader and the RFID tag, and a communication from the at least one reader indicating to activate a new authentication datum.

Clause 66. The medical waste collection system of any one of clauses 57-65, wherein the RFID tag includes a first antenna for receiving a power signal from the at least one reader and a second antenna for communicating the first and second authentication data to the at least one reader.

Clause 67. The medical waste collection system of any one of clauses 57-66, wherein the RFID tag is configured to render the first authentication datum inactive responsive to identifying occurrence of the predefined event.

Clause 68. The medical waste collection system of clause 67, wherein the at least one controller is configured to: responsive to receiving the second authentication data, determine whether the first authentication datum is inactive; and control operation of the medical waste collection device to provide suction based on the determination of whether the first authentication datum is inactive.

Clause 69. The manifold or a method for preparing the manifold of any one of clauses 1-68.

Clause 70. The RFID tag(s) or a method for preparing the RFID tag(s) of any one of clauses 1-68.

What is claimed is:

1. A medical waste collection system comprising:
a medical waste collection device for providing suction at a surgical site; and
a manifold couplable to the medical waste collection device, the manifold defining a pathway through which the medical waste collection device is configured to provide the suction to the surgical site, the manifold including at least one RFID tag comprising a first memory device and a second memory device, the first and second memory devices storing first and second manifold authentication data respectively,
wherein the medical waste collection device comprises:
a waste container for collecting medical waste material;
a receiver in fluid communication with the waste container and dimensioned to removably receive the manifold;
at least one reader disposed adjacent the receiver and configured to communicate with the at least one RFID tag according to a first communications protocol and a second communications protocol when the manifold is inserted in the receiver; and
at least one controller coupled to the at least one reader and configured to:
operate the at least one reader according to the first communications protocol to read the first manifold authentication data from the first memory device of the at least one RFID tag,
operate the at least one reader according to the second communications protocol to read the second manifold authentication data from the second memory device of the at least one RFID tag, and
control operation of the medical waste collection device to provide suction based on the read first and second manifold authentication data.

2. The medical waste collection system of claim 1, wherein the first communications protocol differs from the second communications protocol in at least one of syntax, timing, power level, voltage level, current level, carrier frequency, modulation type, and data encryption algorithm.

3. The medical waste collection system of claim 1, wherein the first communications protocol has at least one of a higher carrier frequency and a lower power level than the second communications protocol.

4. The medical waste collection system of claim 1, wherein the at least one RFID tag comprises a first RFID tag including the first memory device and a second RFID tag including the second memory device, the first and second RFID tags being disposed at different positions of the manifold.

5. The medical waste collection system of claim 4, wherein the manifold comprises a proximal region and a distal region, the proximal region including a port for engaging a vacuum inlet integral with the medical waste collection device when the manifold is inserted in the receiver, and the distal region including a plurality of inlet fittings for engaging suction tubes extending to the surgical site, and wherein the first RFID tag is disposed along an outer surface of the manifold extending from the proximal region to the distal region, and the second RFID tag is disposed at the proximal region adjacent the port.

6. The medical waste collection system of claim 4, wherein the manifold or medical waste collection device comprises a blocking media disposed adjacent the second RFID tag or the at least one reader respectively for preventing the at least one reader from communicating with the second RFID tag according to the first communications protocol.

7. The medical waste collection system of claim 1, wherein the first memory device and second memory device are incorporated in a single RFID tag.

8. The medical waste collection system of claim 7, wherein the single RFID tag includes a first antenna for communication via the first communications protocol and a second antenna for communication via the second communications protocol.

9. The medical waste collection system of claim 8, wherein the single RFID tag includes a first antenna for receiving a power signal from the at least one reader and a second antenna for communicating the first and second manifold authentication data to the at least one reader.

10. The medical waste collection system of claim 1, wherein the at least one controller is configured to communicate a first request for the first manifold authentication data according to the first communications protocol to the at least one RFID tag, the first request including first rover authentication data, and the at least one RFID tag is configured to communicate the first manifold authentication data to the at least one reader based on the first rover authentication data.

11. The medical waste collection system of claim 10, wherein the at least one controller is configured to communicate a second request for the second manifold authentication data according to the second communications protocol to the at least one RFID tag, the second request including second rover authentication data that is different from the first rover authentication data, and the at least one RFID tag is configured to communicate the second manifold authentication data to the at least one reader based on the second rover authentication data.

12. The medical waste collection system of claim 11, wherein the at least one RFID tag is configured to:
apply a first cryptographic algorithm to the first rover authentication data to produce a first output;
communicate the first manifold authentication data to the at least one reader based on the first output;
apply a second cryptographic algorithm, different from the first cryptographic algorithm, to the second rover authentication data to produce a second output; and
communicate the second manifold authentication data to the at least one reader based on the second output.

13. The medical waste collection system of claim 11, wherein the at least one controller is configured to communicate the second request for the second manifold authentication data to the at least one RFID tag based on the first manifold authentication data received from the RFID tag.

14. The medical waste collection system of claim 13, wherein the first manifold authentication data indicates at least one authentication datum for the second request for the second manifold authentication data, the at least one authentication datum indicating at least one of an encryption key for generating the second request, an encryption algorithm for generating the second request, and the second communications protocol for communicating the second request.

15. The medical waste collection system of claim 12, wherein the at least one RFID tag comprises a first processing core and a second processing core, the first processing core being configured to:
communicate the first manifold authentication data to the at least one reader responsive to the at least one RFID tag receiving the first request for the first manifold authentication data that includes the first rover authentication data; and
communicate third manifold authentication data to the second processing core based on the first rover authentication data received from the at least one reader, wherein the second processing core is configured to communicate the second manifold authentication data to the at least one reader based on the second rover authentication data from the at least one reader and the third manifold authentication data from the first processing core.

16. The medical waste collection system of claim 15, wherein the third manifold authentication data indicates at least one authentication datum for verifying the second rover authentication data.

17. The medical waste collection system of claim 1, wherein the at least one controller is configured to:
responsive reading the second manifold authentication data, determine whether the second memory device is inoperable using the at least one reader; and
control operation of the medical waste collection device to provide suction based on the determination of whether the second memory device is inoperable.

18. The medical waste collection system of claim 1, wherein the at least one controller is configured to:
apply a first cryptographic algorithm and a second cryptographic algorithm, different from the first cryptographic algorithm, to the read first and second manifold authentication data respectively to provide first and second outputs respectively; and
control operation of the medical waste collection device to provide suction based on the first and second outputs.

19. A method of preparing an RFID tag for a manifold configured to be coupled to a vacuum inlet integral with a medical waste collection device to provide suction at surgical site through the manifold, the method comprising:
obtaining an RFID tag including a first antenna associated with a first communications protocol, a second antenna associated with a second communications protocol, a first memory device, a second memory device, and a controller coupled to the first and second antennas and the first and second memory devices;
storing first authentication data for being read by the medical waste collection device when the manifold is proximate the medical waste collection device to control actuation of the medical waste collection device in the first memory device; and
storing a second authentication data for being read by the medical waste collection device when the manifold is proximate the medical waste collection device to control actuation of the medical waste collection device in the second memory device,
wherein responsive to the first antenna receiving an authentication data request via the first communications protocol, the controller is configured to communicate the first authentication data via the first antenna, and responsive to the second antenna receiving an authentication data request via the second communications protocol, the controller is configured to communicate the second authentication data via the second antenna.

20. A manifold for connection with a medical waste collection device to provide suction at a surgical site through the manifold, the manifold comprising:
a manifold housing having a proximal region and a distal region and defining an internal fluid pathway between the proximal and distal regions;
a plurality of inlet fittings disposed at the distal region of the manifold housing and each configured to be removably coupled with a suction tube;
an outlet opening disposed at the proximal region of the manifold housing and configured to receive a suction inlet integral with the medical waste collection device for providing suction at the inlet fittings, wherein the outlet opening is in fluid communication with the inlet fittings through the internal fluid pathway defined by the manifold housing;
a first RFID tag including a first memory device storing first manifold authentication data for being read by the medical waste collection device when the manifold is proximate the medical waste collection device to control actuation of the medical waste collection device; and
a second RFID tag including a second memory device storing second manifold authentication data for being read by the medical waste collection device when the manifold is proximate the medical waste collection device to control actuation of the medical waste collection device,
wherein the first RFID tag is disposed along an outer surface of the manifold extending from the proximal region to the distal region, and the second RFID tag is disposed at the proximal region adjacent the outlet opening.
